(19) United States
(12) Patent Application Publication
(10) Pub. No.: US 2024/0159763 A1
(43) Pub. Date: May 16, 2024

(54) DETECTION OF BIOMARKERS USEFUL IN DIAGNOSING CHRONIC ENTEROPATHIES IN CATS

(71) Applicant: VETICA LABS, INC., San Diego, CA (US)

(72) Inventors: Juan ESTRUCH, San Diego, CA (US); Geneviève HANSEN, San Diego, CA (US)

(73) Assignee: VETICA LABS, INC., San Diego, CA (US)

(21) Appl. No.: 18/550,111

(22) PCT Filed: Mar. 11, 2022
(86) PCT No.: PCT/US2022/071112

§ 371 Date: Sep. 11, 2023

Related U.S. Application Data
(60) Provisional application No. 63/159,854, filed on Mar. 11, 2021.

(51) Int. Cl. G01N 33/574; G01N 21/59; G01N 33/58

(52) U.S. Cl. CPC G01N 33/57488; G01N 21/59; G01N 33/581; G01N 2333/4742; G01N 2333/70546; G01N 2333/908

(57) ABSTRACT

The disclosure provides methods and materials for detecting endogenous IgA antibodies to one or more, or all, of OmpC (ACA), Ki67 (AKiA), TK1, integrin (AINTA) and keratin (AKERA), which are useful to diagnose and distinguish chronic enteropathies, e.g. gastrointestinal neoplasms, e.g., gastrointestinal lymphoma, and, inflammatory conditions, e g inflammatory bowel disease, in felines.

### RELATED APPLICATIONS

This application claims the benefit of U.S. Provisional Application Ser. No. 63/159,854, filed Mar. 11, 2021, the contents of which application are incorporated herein by reference.

### FIELD OF THE INVENTION

The invention relates generally to the fields of chronic enteropathies and immunology, for example inflammatory bowel disease and gastrointestinal neoplasia, and more specifically to serological methods and specific algorithms for diagnosing and differentiating chronic enteropathies, such as inflammatory bowel disease and gastrointestinal lymphoma, from other diseases in felines, particularly comprising detecting and measuring endogenous antibodies, as well as diagnostic kits for carrying out such methods, and methods of monitoring disease evolution and response to treatment.

### BACKGROUND OF THE INVENTION

Inflammatory conditions, particularly inflammatory bowel disease, and neoplasms, particularly gastrointestinal neoplasia such as gastrointestinal lymphoma, are identified by veterinarians as common causes of feline chronic enteropathies (CE). Effective treatment of inflammatory conditions such as inflammatory bowel disease and neoplasia, such as gastrointestinal lymphoma, requires differentiating these conditions from each other and from other gastrointestinal disorders.

Inflammatory conditions affecting the gastrointestinal system may have a variety of causes. Inflammatory bowel disease (IBD) describes gastrointestinal disorders that are characterized by persistent or recurrent gastrointestinal (GI) signs and histological evidence of GI inflammation. IBD is classified by anatomic location and by the predominant cell type involved. If the inflammation is localized in the stomach, the small intestine and the colon, the condition is referred to as gastritis, enteritis and colitis, respectively. The most prevalent form of IBD is lymphoplasmacytic enteritis involving lymphocytes and plasma cells invading the small intestine. In some forms of IBD, eosinophils, neutrophils and macrophages may also be part of the inflammatory cells. Furthermore, changes in mucosal architecture, such as villous morphology and fibrosis, may occur and have been reported to possibly correlate to disease severity.

A significant contributor to chronic enteropathies in cats is gastrointestinal (GI) neoplasia. The most common GI neoplasia in cats is represented by GI lymphoma characterized by infiltration of any segments of the GI tract with neoplastic lymphocytes resulting in either segmental or generalized thickening of the tissues. Enteropathy-associated T-cell lymphoma (EATL) type 2, a small cell lymphoma, is the most prevalent form of feline GI lymphoma. Other GI lymphomas such as the large T-cell lymphoma, designated EATL type 1, and B-cell lymphomas may also occur in cats, but are less common.

The etiology of both feline IBD and GI lymphoma remains unclear, but it is believed to be caused by a combination of genetic, environmental, and immune-regulatory factors in susceptible subjects that lead to a broad presentation of clinical signs including vomiting, diarrhea, appetite changes and weight loss. As all these clinical symptoms might be similar for both diseases, definitive diagnosis becomes extremely challenging.

The management of chronic enteropathies includes a variety of treatments that depends on disease locations, severity, and complications. Sequential treatment is commonly recommended with an initial dietary change followed by treatment based on immunosuppression modalities using various steroids and cytotoxic medications such as chlorambucil. In some instances, surgery may be performed for removal of abdominal mass. But most importantly, effective treatment of chronic enteropathies requires differentiation between IBD and GI lymphomas and differentiation from other gastrointestinal disorders. Considering the high incidence of CE, they are likely to affect a significant number of animals during their lifetime, and when left untreated, may lead to an increase of morbidity and deteriorating quality of life. Therefore, prompt and accurate diagnosis of CE is needed to help with rapid and adequate interventions. (Evans et al. J American Vet Medical Association. 229:1447-1450 (2006); Fragkou, F. C., et al. J Vet Internal Medicine, 30: 1031-1045 (2016); Jergens et al. J. Vet Internal Medicine, vol. 24, no. 5, 2010, pp. 1027-1033; Jergens, J Feline Med Surg. 14:445-58 (2012); Kiupel et al., Vet Pathology, 48:212-222 (2010); Lamb et al., J Crohn's and Colitis, 12: S653-S668 (2018); Makielski et al., J Vet Internal Medicine. 33:11-22(2018); Paulin et al. BMC Vet Research. 14:306 (2018); Marsilio et al. J Vet Internal Medicine. 33:551-558(2019); Moore et al., Vet. Pathol. 49:658-668(2012); Norsworthy et al., J American Vet Medical Association. 243:1455-1461 (2013); Scott et al., J Vet Internal Medicine, 25: 1253-1257 (2011)).

Current methods to diagnose chronic enteropathies and to differentiate IBD from GI neoplasia in cats requires relatively costly, labor-intensive and intrusive clinical techniques with the current gold standard test consisting of histopathologic examination of tissue biopsies collected under general anesthesia. Even then, the final diagnosis remains complicated because the cells involved in mucosal changes of IBD and GI neoplasia may be morphologically indistinguishable and may be unevenly distributed throughout the GI tract. Furthermore, the pathological lesions can be inflammatory or neoplastic in nature and these two forms may coexist. It is generally believed that inflammatory lesions progress to neoplastic lesions. In an attempt to resolve this ambiguity, histopathologic assessment is often followed by immunohistochemistry and clonality testing of three or more full-thickness biopsy specimens collected from locations believed to containing pathological lesions. And despite all these techniques, there may still exist a high degree of subjectivity. Therefore, less invasive and more accurate diagnostic modalities would be highly desirable.

US20170248614A1, incorporated herein by reference, describes methods of diagnosing inflammatory bowel disease in dogs, as well as assays for the detection of endogenous antibodies to antigens associated with food sensitivities, to inflammatory markers in dogs, and to bacteria from the canine microbiome, but does not identify methods of distinguishing IBD from GI neoplasia in felines.

The availability of rapid and less intrusive methods to identify IBD patients and/or GI neoplasia patients and to differentiate IBD from GI neoplasia in felines would represent a major clinical advance in veterinary medicine and would facilitate earlier and more appropriate intervention to treat diseased pets as well as to monitor disease evolution and response to treatment. There is a need for a more effective, less intrusive approach tailored specifically for cats. The present invention satisfies these needs and provides related advantages as well.

### BRIEF DESCRIPTION OF THE INVENTION

The present invention provides novel markers and methods for detecting chronic enteropathies in cats, to aid in diagnosis and monitoring of inflammatory diseases and neoplasia, either on a systemic basis and/or on a localized basis such as in the gastrointestinal tract. Endogenous antibodies have been identified which correlate with development of neoplasms, particularly GI neoplasia, and with inflammatory conditions in cats. Measuring the presence and level of these endogenous antibodies in cats is extremely useful for early detection and diagnosis of these conditions, thereby allowing for early and appropriate treatment.

In one aspect, the present invention provides a method for classifying whether a cat is suffering from an inflammatory condition, e.g., IBD, the method comprising: (a) determining the presence or level of at least one marker selected from the group consisting of antimicrobial antibody, anti-inflammation antibody, anti-wound repair antibody, anti-proliferation antibody and combinations thereof in the sample; and (b) classifying the sample as consistent with IBD or non-consistent with IBD sample using a statistical algorithm based upon the presence or level of at least one marker.

In another aspect, the present invention provides a method for classifying whether a cat is suffering from a GI neoplastic diseases such as lymphoma and the like, the method comprising: (a) determining the presence or level of at least one marker selected from the group consisting of antimicrobial antibody, anti-inflammation antibody, anti-wound repair antibody, anti-proliferation antibody and combinations thereof in the sample; and (b) classifying the sample as consistent with GI neoplasia or non-consistent with GI neoplasia sample using a statistical algorithm based upon the presence or level of at least one marker.

In yet another aspect, the present invention provides a method for differentiating whether a cat exhibiting GI symptoms, such as diarrhea, is suffering from an inflammatory condition, e.g. IBD or a neoplasm, e.g., GI lymphoma, the method comprising: (a) determining the level of at least one marker selected from the group consisting of antimicrobial antibody, anti-inflammation antibody, anti-wound repair antibody, anti-proliferation antibody and combinations thereof in the sample; and (b) classifying the sample as consistent with inflammation, e.g., IBD, or consistent with a neoplasm, e.g., GI Lymphoma, or non-consistent with an inflammatory condition, e.g., IBD, or non-consistent with a neoplasm, e.g., GI Lymphoma, using a statistical algorithm based upon the level of at least one marker.

In the case of inflammatory conditions such as IBD in felines, the present invention describes certain novel types of endogenous antibodies to microbes found in the gut and endogenous antibodies to inflammation markers, to wound repair markers, and to proliferation markers. In certain embodiments, measuring levels of endogenous antibodies to microbes found in the gut markers is coupled with measurement of one or more other markers of inflammation, wound repair and proliferation.

In the case of gastrointestinal neoplastic diseases in felines, the present invention describes certain novel types of endogenous antibodies to microbes found in the gut and endogenous antibodies to inflammation markers, to wound repair markers, and to proliferation markers. In certain embodiments, measuring levels of endogenous antibodies to microbes found in the gut markers is coupled with measurement of one or more other markers of inflammation, wound repair and proliferation.

For example, it has surprisingly been discovered that feline animals, when suffering from inflammatory conditions, produce autoantibodies to proteins such as β-integrins and/or calprotectin, which are known to be associated with inflammation. Such autoantibodies have not previously been discovered or characterized, and it is unexpected and counter-intuitive that the body would produce antibodies to its own inflammatory proteins, or that such antibodies could serve as markers for pathological inflammatory conditions.

In another example, it has surprisingly been discovered that feline animals, when suffering from wound repair conditions produce autoantibodies to proteins such as keratin 18 and TIMP (tissue inhibitors of metalloproteinases), which are known to be associated with wound repair. Such autoantibodies have not previously been discovered or characterized, and it is unexpected and counter-intuitive that the body would produce antibodies to its own wound repair proteins, or that such antibodies could serve as markers for pathological inflammatory conditions.

In yet another example, it has surprisingly been discovered that feline animals, when suffering from neoplastic diseases, such as lymphoma, produce autoantibodies to proteins such as Ki67 and/or TK1, which are known to be associated with cell proliferation. Such autoantibodies have not previously been discovered or characterized, and it is unexpected and counter-intuitive that the body would produce antibodies to its own proliferation proteins, or that such antibodies could serve as markers for pathological neoplastic conditions.

The presence, absence, and/or level of the endogenous antibodies to microbes found in the gut and endogenous antibodies to inflammation markers, to wound repair markers, and to proliferation markers, as described herein, is shown to correlate with the disease state of the cat, allowing early detection and differentiation of an inflammatory condition such as IBD from a neoplastic condition such as GI lymphoma, without initially requiring more invasive and expensive approaches such as tissue biopsies collected under general anesthesia.

In a related aspect, the present invention provides a method for monitoring disease evolution and/or treatment response in a feline receiving treatment for chronic enteropathies, e.g., inflammatory conditions such as IBD and/or neoplasms such as GI lymphoma, the method comprising: (a) determining the presence or level of at least one marker selected from the group consisting of an antimicrobial antibody, anti-inflammation antibody, anti-wound repair, anti-proliferation and combinations thereof in a sample from the individual; and (b) differentiating between an inflammatory condition, e.g., IBD and/or a neoplastic condition, e.g., GI lymphoma, in the feline using a statistical algorithm based upon the presence or level of the at least one marker. Notably, the methods of the invention provide a means for detecting and monitoring disease evolution and/or treatment response in a feline where tissue biopsies may be impractical or uninformative. Not only are tissue biopsies invasive and expensive, but the pathology of the disease, including scarring and other changes to the tissue, may alter the tissue so that it can no longer be usefully compared to normal tissue, making histological analysis alone inadequate to determine whether or not the disease is in remission.

Thus, in accordance with the methods of the present invention, the level of the different markers in a sample from cats believed to be suffering from chronic enteropathies, e.g., IBD and/or GI neoplasia, is determined and compare to the presence or absence of the same markers in non-IBD and/or non-GI neoplasia cats. The methods of the present invention are performed using immunochemical reagents, for example, to detect endogenous antimicrobial antibodies, anti-inflammation antibodies, anti-wound repair, and -proliferation and the like. Thus, there is an array of different immunoassay formats in which the methods of the present invention may be performed. Also provided by the present invention are kits for screening feline IBD and/or GI neoplasia. Suitable kits include immunochemical reagents useful for determining certain endogenous antibodies in a sample.

In certain instances, the methods and systems of the present invention compose a step having a “transformation” or “machine” associated therewith. For example, an ELISA technique may be performed to measure the presence or concentration level of many of the markers described herein. An ELISA includes transformation of the marker, e.g., an endogenous-antibody, into a complex between the marker (e.g., the endogenous antibody) and a binding agent (e.g., antigen), which can then be measured with a labeled secondary antibody. In many instances, the label is an enzyme which transforms a substrate into a detectable product. The detectable product measurement can be performed using a plate reader such as a spectrophotometer. In other instances, genetic markers are determined using various amplification techniques such as PCR. Method steps including amplification such as PCR result in the transformation of single or double strands of nucleic acid into multiple strands for detection. The detection can include the use of a fluorophore, which is performed using a machine such as a fluorometer.

Further areas of applicability of the present invention will become apparent from the detailed description provided hereinafter. It should be understood that the detailed description and specific examples, while indicating certain embodiments of the invention, are intended for purposes of illustration only and are not intended to limit the scope of the invention.

### DETAILED DESCRIPTION OF THE INVENTION

The following description of different embodiments is merely exemplary in nature and is in no way intended to limit the invention, its application, or uses.

### I. Definitions

As used herein, the following terms have the meanings ascribed to them unless specified otherwise.

As used herein, the term “antibody” includes a population of immunoglobulin molecules, which can be polyclonal or monoclonal and of any class and isotype, or a fragment of an immunoglobulin molecule. There are five major classes of immunoglobulins: IgA, IgD, IgE, IgG, and IgM, and several of these may be further divided into subclasses (isotypes), e.g., IgG1, IgG2, IgG3, IgG4, IgA1 (human), IgA2 (human), IgAa (canine), IgAb (canine), IgAc (canine), IgAd (canine), IgA (feline), and IgG (feline). Such fragment generally comprises the portion of the antibody molecule that specifically binds an antigen. For example, a fragment of an immunoglobulin molecule known in the art as Fab, Fab′ or F(ab′)2 is included within the meaning of the term antibody.

As used herein, the term “endogenous antibodies” refers to antibodies made by or originating from the patient, which can be isolated from the patient's blood or tissue. Typically, endogenous antibodies are generated in response to a foreign antigen, for example in response to a bacterial antigen, as part of the body's natural defense against infection. Where the patient is a cat, the endogenous antibodies would obviously be feline antibodies.

The term “endogenous antibodies” is used herein to distinguish from therapeutic or diagnostic antibodies, derived from a source other than the patient, which may for example be administered to the patient or used to detect the presence of antigens in a biological sample (e.g., blood, plasma, urine, tissue, saliva, etc.) from the patient. Therapeutic or diagnostic antibodies would typically be monoclonal antibodies propagated in cell lines, usually derived from antibodies made in other species, e.g., from rodents, or using phage display techniques. Therapeutic antibodies could be complete antibodies or antibody fragments.

“Autoantibody”, as used herein, refers to an endogenous antibody made by the patient against an endogenous antigen, for example against an endogenous protein. The examples herein, for example, describe autoantibodies against endogenous inflammation, wound repair, proliferation-related proteins such as β-integrins, keratin, TK1, and Ki67. Accordingly, where the autoantibody binds to an inflammation-related protein, both the autoantibody and the inflammation-related protein antigen would be from the same individual and the same species, e.g., where the patient is a cat, the autoantibodies generated by the patient are feline antibodies, and the endogenous antigen would be a feline peptide, e.g., feline integrin or feline keratin or feline Ki67 or feline TK1. The autoantibody in such a case can be isolated and characterized by its binding to a protein having the same binding epitope as the endogenous antigen.

As used herein the term “antigen” is understood to be any substance capable of stimulating antibody production. Also, the term “immunogen” is understood to include any substance used to induce an immune response.

The term “autoantigen” refers to a constituent of self that binds an autoantibody or that induces a cellular response.

The term “seropositive” refers to subjects showing a significant level of serum antibodies or other immunological markers indicating previous exposure to immune reactive agent being tested.

“Inflammation” or “inflammatory condition” as used herein refers to an immunovascular response to a stimuli, for example an immune response to an antigen, a pathogen, or a damaged cell, which is mediated by white blood cells (leukocytes). In some embodiments, the inflammation may be chronic. In some embodiments, the inflammation may be an autoimmune condition, where the immune system causes damage to otherwise normal, non-foreign tissue, as is seen for example in rheumatoid arthritis, multiple sclerosis, and other autoimmune diseases. In some embodiments, inflammation may be associated with the migration and accumulation of immune cells in the gastrointestinal tract. In certain embodiments, the gut mucosal homing heterodimeric cell surface receptor α4β7 integrin is involved in the recruitment of immune cells to the intestinal tissues and perpetuation of recurrent inflammation by enabling adhesion, proliferation, and cell-cell and cell-extracellular matrix signaling interactions. In certain aspect, α4β7 can mediate T-cell and/or B-cell lymphocyte migration into the intestinal lamina mucosa leading to marked epitheliotropism with prominent intraepithelial nests and/or plaques of lymphocytes.

The term “inflammatory bowel disease” or “IBD” refers to a chronic inflammation of all or part of the gastrointestinal tract, include, without limitation, the following sub-types: lymphocytic enteritis, lymphocytic gastritis, lymphocytic colitis, lymphoplasmacytic enteritis, lymphoplasmacytic gastritis, lymphoplasmacytic colitis, eosinophilic gastroenteritis, and granulomatous enteritis Inflammatory bowel diseases are distinguished from all other disorders, syndromes, and abnormalities of the gastroenterological tract, including transient GI infections, in being characterized by chronic inflammation.

“Neoplasia” and “neoplastic diseases” and “neoplastic conditions” and “cancer” and “cancerous” refer to or describe the physiological condition in mammals that is typically characterized by unregulated cell growth. Unregulated cell growth can lead to the formation of malignant tumors that infiltrate adjacent tissues and may also metastasize to distant parts of the body. Examples of neoplasia include, but are not limited to lymphoma, mast cell tumor, sarcoma, or lymphoid malignancies. Neoplasia as it applies to a subject diagnosed with, or suspected of having, a neoplasia may have a malignant or potentially malignant neoplasm or tissue mass of any size, and includes primary tumors and secondary neoplasms. Commonly involved sites of neoplasia in cats are gastrointestinal neoplasia with gastrointestinal lymphoma being the most prevalent.

The term “wound repair” refers to a complex and dynamic interaction of biological processes including inflammation, proliferation, remodeling of connective tissue resulting in increased levels of inflammatory cells, proteases, and some structural proteins (e.g. extracellular matrix and the likes). In some aspect, persistent inflammation involves a cascade of mediators capable of inducing lesions and modifications in the GI wall structure. In some aspect, recurrent inflammatory influx is accompanied by extensive remodeling of epithelial connective tissues with aberrant deposit of extracellular matrix resulting in intestinal fibrosis. In some aspect, cytoplasmic constituents associated with epithelial integrity and cell organization maintenance, like keratin, may be involved in recurrent chronic inflammation associated with IBD and GI neoplasia.

The term “proliferation” or “cell proliferation” refers to unregulated cell growth or uncontrolled cell growth associated with various factors. Proliferation can be detected by various means, e.g., metabolic activity assays (e.g., increases in lactate dehydrogenase activity during proliferation as measured by tetrazolium salts or Alamar Blue assay), cell proliferation marker assays (e.g., immunoassays to detect proliferation markers such as Ki67, TK1, proliferating cell nuclear antigen PCNA, topoisomerase IIB, and/or phosphorylated histone H3), ATP concentration assays (e.g., using luciferase activities), and DNA synthesis assays (e.g., detecting e bromodeoxyuridine (BrdU) incorporation into DNA). In some aspects, the factor associated with proliferation is a nuclear protein like Ki67, which is detected during all active phases of cell cycle (G1, S, G2, and mitosis) and is strongly associated with tumor cell proliferation. In yet another aspect, the factor is an enzyme protein like thymidine kinase 1 (TK1) detected during the S/G2 phases of cell cycle when DNA synthesis occurs. In some aspects TK1 and/or Ki67 is released from cells that die and disrupt during proliferation. Thus, the concentration of TK1 and/or Ki67 in extracellular fluid is a measure of disruption of dividing cells, particularly during accelerated or unregulated proliferation, as in the case of malignancy. Moreover, such TK1 and/or Ki67 can result in generation of auto-antibodies, e.g. IgA auto-antibodies to TK1 and/or Ki67. Thus in one aspect, “proliferation” or “cell proliferation” can refer to proliferation characterized or detected by the presence of autoantibodies to Ki67 and/or autoantibodies to TK1, e.g. as detected in extracellular fluid, e.g., from blood, or serum e.g., using an immunoassay, e.g., an ELISA assay.

“Microbes found in the gut” refer to intestinal microbiota that is defined as the aggregate of all live micro-organisms that inhabit the gastrointestinal tract. The gastrointestinal tract of animals is colonized by a heterogenous group of microorganisms known as GI microbiota. In monogastric animals the intestine contains the most abundant, diverse, and metabolically relevant group of bacteria in the GI tract. For instance, the feline intestinal tract is colonized by a vast assortment of commensal microbial species with surface antigens that can potentially stimulate strong immune response in a susceptible host leading to the expression of high titers of specific antibodies to those particular antigens. In the case of feline chronic enteropathies, the fecal microbial communities may be increased with members of particular taxa, for instance members of the Enterobacteriaceae, Streptococcaceae, and Pseudomonadaceae taxa.

The term “food sensitivity” refers to an immune-mediated reaction to food arising from immune responses that occur reproducibly on exposure to a given set of epitopes derived from food. The reactive immune response is characterized as types I, II, III or IV depending on the mechanism involved and the delayed nature of such response. Food sensitivity may involve types II, III or IV in which more complex set of immune cells are involved and may take between hours and weeks between the exposure and the response, and such response can be chronic in nature.

The term “sample” includes any biological specimen obtained from a cat. Suitable samples for use in the present invention include, without limitation, whole blood, plasma, serum, saliva, urine, stool, tears, any other bodily fluid, tissue samples (e.g., biopsy), and cellular extracts thereof (e.g., red blood cellular extract). The use of samples such as serum, saliva, and urine is well known in the art (Hashida et al. J. Clin. Lab. Anal., 11:267-286 (1997). One skilled in the art will appreciate that samples such as serum samples can be diluted prior to the analysis of marker levels.

The term “marker” includes any biochemical marker, serological marker, genetic marker, or other clinical or echographic characteristic that can be used to classify a sample from a cat as being associated with an inflammatory condition and/or neoplasia, such as IBD and lymphoma. For instance, serologic markers are important in chronic enteropathies because their expression represents the host response to antigens exposed as a result of a combination of pathophysiological processes resulting in the breakdown of the gut mucosal barrier and a primed and sometime even over-reactive immune system. Similarly, the presence of active gut inflammation is associated with migration of leukocytes to the gut, and elevates multiple proteins detectable in serum, plasma, and/or feces. Non-limiting examples of markers suitable for use in the present invention include anti-PMN antibodies of the IgA classification (e.g., APMNA, pAPMNA, cAPMNA, ANSNA, ASAPPA, and the like), antimicrobial antibodies (e.g., anti-Outer-Membrane Protein, anti-OmpC antibodies (ACA), anti-flagellin antibodies (AFA), and the like), and the like and combinations thereof, anti-food-related antigen antibodies (e.g., anti-gliadin antibodies (AGA), anti-zein antibodies (AZA), and the like), as well as autoantibodies to endogenous proliferation related-proteins such as Ki67 (AKiA) or TK1, autoantibodies to endogenous wound repair related-proteins such as Keratin (AKERA), and autoantibodies to inflammation such as integrin (AINTA) and others like calprotectin, lactoferrin, and/or CRP. The recitation of specific examples of markers associated with chronic enteropathies is not intended to exclude other markers as known in the art and suitable for use in the present invention.

The term “marker profile” includes one, two, three, four, five, six, seven, eight, nine, ten, or more diagnostic and/or prognostic marker(s), wherein the markers can be a serological marker, a protein marker, a genetic marker, and the like. In some embodiments, the marker profile together with a statistical analysis can provide veterinarians valuable diagnostic and prognostic insight. In other embodiments, the marker profile with optionally a statistical analysis provides a tool to monitor disease evolution and/or treatment response to a given therapy. Combining information from multiple diagnostic predictors is often useful, because combining data on multiple markers may provide a more sensitive and discriminating tool for diagnosis or screening applications than any single marker on its own. By using multiple markers (e.g., serological, protein, genetic, etc.) in conjunction with statistical analyses, the assays described herein provide diagnostic, prognostic and therapeutic value by identifying patients with chronic enteropathies or a clinical subtype thereof such as IBD or GI neoplasia, and or with food sensitivities or clinical subtype thereof, predicting risk of developing complicated disease, assisting in assessing the rate of disease progression (e.g., rate of progression to complicated disease or surgery), and assisting in the monitoring of condition.

The term “label,” as used herein, refers to a detectable compound, composition, or solid support, which can be conjugated directly or indirectly (e.g., via covalent or non-covalent means, alone or encapsulated) to a monoclonal antibody or a protein. The label may be detectable by itself (e.g., radioisotope labels, chemiluminescent dye, electrochemical labels, metal chelates, latex particles, or fluorescent labels) or, in the case of an enzymatic label, may catalyze chemical alteration of a substrate compound or composition which is detectable (e.g., enzymes such as horseradish peroxidase, alkaline phosphatase, and the like). The label employed in the current invention could be, but is not limited to alkaline phosphatase; glucose-6-phosphate dehydrogenase (“G6PDH”); horseradish peroxidase (HRP); chemiluminescers such as isoluminol, fluorescers such as fluorescein and rhodamine compounds; ribozymes; and dyes. The label may also be a specific binding molecule which itself may be detectable (e.g., biotin, avidin, streptavidin, digioxigenin, maltose, oligohistidine, e.g., hex-histidine, 2, 4-dinitrobenzene, phenylarsenate, ssDNA, dsDNA, and the like). The utilization of a label produces a signal that may be detected by means such as detection of electromagnetic radiation or direct visualization, and that can optionally be measured.

A monoclonal antibody can be linked to a label using methods well known to those skilled in the art, e.g., Immunochemical Protocols; Methods in Molecular Biology, Vol. 295, edited by R. Bums (2005)). For example, a detectable monoclonal antibody conjugate may be used in any known diagnostic test format like ELISA or a competitive assay format to generate a signal that is related to the presence or amount of an IBD-associated antibody and/or is related to the presence or amount of a food sensitivity-associated antibody and/or is related to presence or amount of a chronic enteropathy-associated antibody in a test sample.

“Substantial binding” or “substantially binding” refer to an amount of specific binding or recognizing between molecules in an assay mixture under particular assay conditions. In its broadest aspect, substantial binding relates to the difference between a first molecule's incapability of binding or recognizing a second molecule, and the first molecules capability of binding or recognizing a third molecule, such that the difference is sufficient to allow a meaningful assay to be conducted to distinguish specific binding under a particular set of assay conditions, which includes the relative concentrations of the molecules, and the time and temperature of an incubation. In another aspect, one molecule is substantially incapable of binding or recognizing another molecule in a cross-reactivity sense where the first molecule exhibits a reactivity for a second molecule that is less than 25%, e.g. less than 10%, e.g., less than 5% of the reactivity exhibited toward a third molecule under a particular set of assay conditions, which includes the relative concentration and incubation of the molecules. Specific binding can be tested using a number of widely known methods, e.g., an immunohistochemical assay, an enzyme-linked immunosorbent assay (ELISA), a radioimmunoassay (RIA), or a western blot assay.

The term “patient” or “subject” in the context of this application refers to domestic cats.

As used herein, the term “substantially the same amino acid sequence” includes an amino acid sequence that is similar, but not identical to, the naturally-occurring amino acid sequence. For example, an amino acid sequence, i.e., polypeptide, that has substantially the same amino acid sequence as an Outer-Membrane Protein (OmpC) protein can have one or more modifications such as amino acid additions, deletions, or substitutions relative to the amino acid sequence of the naturally-occurring OmpC protein, provided that the modified polypeptide retains substantially at least one biological activity of OmpC such as immunoreactivity. The “percentage similarity” between two sequences is a function of the number of positions that contain matching residues or conservative residues shared by the two sequences divided by the number of compared positions times 100. In this regard, conservative residues in a sequence is a residue that is physically or functionally similar to the corresponding reference residue, e.g., that has a similar size, shape, electric charge, chemical properties, including the ability to form covalent or hydrogen bonds, or the like. Sequence similarity algorithms used by BLAST, FASTA, SSEARCH, and other popular similarity searching programs produce accurate statistical estimates of sequence similarity and are widely used to search, rank, and predict the function of proteins based on their degree of similarity to other proteins. Additionally, as discussed below, software to predict antigenicity of amino acid sequences is widely available, meaning that sequences can be readily designed which contain some variation from the natural sequence, while conserving the antigenic features of the original protein.

“Amino acid consensus sequence,” as used herein, refers to a hypothetical amino acid sequence that can be generated using a matrix of at least two, for example, more than two, aligned amino acid sequences, and allowing for gaps in the alignment, such that it is possible to determine the most frequent amino acid residue at each position. The consensus sequence is that sequence which comprises the amino acids which are most frequently represented at each position. In the event that two or more amino acids are equally represented at a single position, the consensus sequence includes both or all of those amino acids. In some cases, amino acid consensus sequences correspond to a sequence or sub-sequence found in nature. In other cases, amino acid consensus sequences are not found in nature, but represent only theoretical sequences.

“Homology” is an indication that two nucleotide sequences represent the same gene or a gene product thereof, and typically means that that the nucleotide sequence of two or more nucleic acid molecules are partially, substantially or completely identical. When from the same organism, homologous polynucleotides are representative of the same gene having the same chromosomal location, even though there may be individual differences between the polynucleotide sequences (such as polymorphic variants, alleles and the like).

The term “heterologous” refers to any two or more nucleic acid or polypeptide sequences that are not normally found in the same relationship to each other in nature. For instance, a heterologous nucleic acid is typically recombinantly produced, having two or more sequences, e.g., from unrelated genes arranged to make a new functional nucleic acid, e.g., a promoter from one source and a coding region from another source. Similarly, a heterologous polypeptide will often refer to two or more subsequences that are not found in the same relationship to each other in nature (e.g., a fusion protein).

As used herein, the term “fragment” includes a peptide, polypeptide or protein segment of amino acids of the full-length protein, provided that the fragment retains reactivity with at least one antibody in sera of disease patients. In some embodiments, the antigen or fragment thereof comprises at the amino-terminus and/or carboxyl-terminus one or more or a combination of fusion tags, e.g., to enhance solubility and/or purification of the antigen, for example tags such as a polyhistidine tag (e.g., 6×His tag) which has a strong affinity to transition metal ions, e.g. Ni²⁺, though Co²⁺, Cu²⁺, and Zn²⁺; a Small Ubiquitin-like Modifier (SUMO) tag, which enhances solubility, and may be selectively cleaved by a SUMO protease; a glutathione S-transferase (GST) tag, which enhances solubility and has a strong affinity to the reduced form of glutathione (GSH)); and the like.

An “antigenic fragment” is a fragment of a full-length protein that comprises an antibody binding epitope, for example an epitope to which an antibody of interest exhibits substantial binding. It is not necessary that the antigen capture every endogenous antibody to a protein of interest, so long as the antigen will capture a sufficient proportion of the endogenous antibodies to reliably measure the presence, absence and/or level of the endogenous antibodies of interest in the sample. Therefore, it is generally not required to use a full-length protein as the antigen, but where fragments or variants of antigens are made for use herein, it is preferable to conserve the antigenic regions of the proteins, i.e., regions which are likely to comprise one or more binding epitopes for the antibodies of interest. The antigenicity of an amino acid sequence can be readily predicted, as it is known that certain residues or combinations of residues are far more likely to serve as antibody epitopes than others. Software can be used to predict antigenicity of an amino acid sequence in a protein. Typically, such software uses algorithms to score the antigenicity potential of a given sequence, to identify portions of the sequence which are likely to contain epitopes and for example indicate the amino acid residue with the highest antigenicity properties within the context of the given amino acid sequence. Such tools include SVMTriP (http://susbio.unl.edu/SVMTriP/), iMed (http://imed.med.ucm.es/Tools/antigenic.pl), Emboss (http://bioinformatics.nl/cgi-bin/emboss/antigenic), EpiC (http://bioware.ucd.ie/epic/), as well as more sophisticated commercial programs like OptimumAntigen™ Design Tool by GenScript. At the same time, the antigens used herein may comprise fusion tags to facilitate isolation or solubility, e.g., polyhistidine, SUMO, or GST tags and the like, as further described herein. For example, the sequences may comprise an antigenic fragment capable of specifically binding to the antibody of interest (e.g. SEQ ID NOS: 1, 4, 7, 10 or 18 or antigenic fragments or variants thereof) which is linked to a fusion tag comprising a polyhistidine sequence for purification, a SUMO tag for enhanced solubility, and a tryptophan residue to enable easy spectrophotometric detection. These same techniques can help ensure that such fusion tags are either bound to substrate and unavailable for binding to antibodies in the sample or do not have antigenic regions that bind unwanted antibodies in the sample or otherwise disrupt the assay.

“Polyhistidine tag” includes any histidine-rich sequence which facilitates purification of a protein, e.g., on a metal substrate, e.g., 6×His or HexaHis tag (e.g., comprising six histidine residues, e.g. as in SEQ ID NO: 13 or 14); an HQ tag having alternating histidine and glutamine (e.g., comprising HQHQHQ) residues; HN tag having alternating histidine and asparagine (e.g., comprising HNHNHNHNHNHN); and a histidine affinity tag (HAT) derived from chicken lactate dehydrogenase, e.g., comprising KDHLIHNVHKEEHAHAHNK.

An “epitope” is the antigenic determinant on a polypeptide that is recognized for binding by a paratope on antibodies specific to the polypeptide, for example, an IBD-associated antibody. Antibodies in the context of the invention may recognize particular epitopes having a sequence of 3 to 11, e.g., 5 to 7, amino acids. The antibody may further be characterized by its binding affinity to the protein, polypeptide or peptide applied in the methods and kits of the invention, and the binding affinity (K_{D}) is, for example, in the nanomolar range, e.g., K_{D} 10⁻⁷ or less, for example, to K_{D} 10⁻⁹ to 10⁻¹⁰. The invention herein detects polyclonal populations of endogenous antibodies in a sample, e.g., serum, e.g., antimicrobial antibody, antiinflammation autoantibody, anti-wound repair autoantibody, antiproliferation autoantibody, and combinations thereof described herein, e.g., endogenous IgA antibodies to one or more, or all, of OmpC (ACA), Ki67 (AKiA), TK1, integrin (AINTA) and keratin (AKERA). Monoclonal or polyclonal antibodies directed against feline antibodies, e.g., specific for feline IgA constant regions, are used as detection antibodies and so may be labeled.

The term “clinical factor” includes a symptom in a patient that is associated with IBD. Examples of clinical factors include, without limitation, diarrhea, abdominal pain and/or discomfort, cramping, fever, anemia, hypoproteinemia, weight loss, anxiety, lethargy, and combinations thereof. In some embodiments, a diagnosis of IBD is based upon a combination of analyzing the presence or level of one or more markers in a patient using statistical algorithms and determining whether the patient has one or more clinical factors.

The term “clinical factor” includes a symptom in a patient that is associated with GI neoplasia. Examples of clinical factors include, without limitation, diarrhea, abdominal pain and/or discomfort, cramping, fever, anemia, hypoproteinemia, weight loss, anxiety, lethargy, and combinations thereof. In some embodiments, a diagnosis of GI neoplasia is based upon a combination of analyzing the presence or level of one or more markers in a patient using statistical algorithms and determining whether the patient has one or more clinical factors.

The term “prognosis” includes a prediction of the probable course and outcome of IBD or the likelihood of recovery from the disease. In some embodiments, the use of statistical algorithms provides a prognosis of chronic enteropathy, e.g. IBD, in a cat. For example, the prognosis can be surgery, development of a clinical subtype of IBD, development of one or more clinical factors, development of intestinal cancer, or remission or recovery from the disease.

The term “prognosis” also includes a prediction of the probable course and outcome of GI neoplasia or the likelihood of recovery from the disease. In some embodiments, the use of statistical algorithms provides a prognosis of GI neoplasia in a cat. For example, the prognosis can be surgery, development of a clinical subtype of GI neoplasia, development of one or more clinical factors, or remission recovery from the disease.

The term “prognostic profile” includes one, two, three, four, five, six, seven, eight, nine, ten, or more marker(s) of a cat, wherein the marker(s) can be a serological marker, a protein marker, a genetic marker, and the like. A statistical analysis transforms the marker profile into a prognostic profile. An example of statistical analysis can be defined, but not limited to, analysis by quartile scores and the quartile score for each of the markers can be summed to generate a quartile sum score.

The term “diagnosing lymphoma” includes the use of the methods, systems, and code of the present invention to determine the presence or absence of lymphoma in a pet patient. The term also includes methods, systems, and code for assessing the level of disease activity in a pet patient.

Detection of the endogenous antibodies described herein is useful for monitoring the progression or regression of lymphoma, including the use of the methods, systems, and code of the present invention to determine the disease state (e.g., presence of cancer, presence of lymphoma) of a feline patient. In certain instances, the results of a statistical algorithm are compared to those results obtained for the same pet patient at an earlier time. In some aspects, the methods, systems, and code of the present invention can also be used to predict the progression of lymphoma, e.g., by determining a likelihood for cancer to progress either rapidly or slowly in a feline based on the presence or level of at least one marker in a sample. In other aspects, the methods, systems, and code of the present invention can also be used to predict the regression of lymphoma, e.g., by determining a likelihood for lymphoma to regress either rapidly or slowly in a pet patient based on the presence or level of at least one marker in a sample.

Detection of the endogenous antibodies described herein is useful for diagnosing cancer or diagnosing neoplasia in a feline, including the use of the methods, systems, and code of the present invention to determine the presence or absence of cancer in a feline patient. The term also includes methods, systems, and code for assessing the level of disease activity in a pet patient. The term “monitoring the progression or regression of cancer” includes the use of the methods, systems, and code of the present invention to determine the disease state (e.g., presence of cancer, presence of cancer) of a pet patient. In certain instances, the results of a statistical algorithm are compared to those results obtained for the same pet patient at an earlier time. In some aspects, the methods, systems, and code of the present invention can also be used to predict the progression of cancer, e.g., by determining a likelihood for cancer to progress either rapidly or slowly in a pet based on the presence or level of at least one marker in a sample. In other aspects, the methods, systems, and code of the present invention can also be used to predict the regression of cancer, e.g., by determining a likelihood for cancer to regress either rapidly or slowly in a pet patient based on the presence or level of at least one marker in a sample.

Detection of the endogenous antibodies described herein is useful for diagnosing IBD, including the use of the methods, systems, and code of the present invention to determine the presence or absence of IBD in a cat. The term also includes methods, systems, and code for assessing the level of disease activity in a cat. The term “monitoring the progression or regression of IBD” includes the use of the methods, systems, and code of the present invention to determine the disease state (e.g., presence or severity of IBD) of a cat. In certain instances, the results of a statistical algorithm are compared to those results obtained for the same cat at an earlier time. In some aspects, the methods, systems, and code of the present invention can also be used to predict the progression of IBD, e.g., by determining a likelihood for IBD to progress either rapidly or slowly in a cat based on the presence or level of at least one marker in a sample. In other aspects, the methods, systems, and code of the present invention can also be used to predict the regression of IBD, e.g., by determining a likelihood for IBD to regress either rapidly or slowly in a cat based on the presence or level of at least one marker in a sample.

Detection of the endogenous antibodies described herein is useful for diagnosing an inflammatory condition, including the use of the methods, systems, and code of the present invention to determine the presence or absence of an inflammatory condition in a cat. The term also includes methods, systems, and code for assessing the level of disease activity in the patient. The term “monitoring the progression or regression of inflammation” includes the use of the methods, systems, and code of the present invention to determine the disease state (e.g., presence or severity of inflammation) of the patient. In certain instances, the results of a statistical algorithm are compared to those results obtained for the same patient at an earlier time. In some aspects, the methods, systems, and code of the present invention can also be used to predict the progression of inflammation, e.g., by determining a likelihood for the inflammation to progress either rapidly or slowly in the patient based on the presence or level of at least one marker in a sample. In other aspects, the methods, systems, and code of the present invention can also be used to predict the regression of inflammation, e.g., by determining a likelihood for inflammation to regress either rapidly or slowly in the patient based on the presence or level of at least one marker in a sample.

Detection of the endogenous antibodies described herein is useful for diagnosing GI neoplasia, including the use of the methods, systems, and code of the present invention to determine the presence or absence of GI neoplasia in a cat. The term also includes methods, systems, and code for assessing the level of disease activity in a cat. The term “monitoring the progression or regression of GI neoplasia” includes the use of the methods, systems, and code of the present invention to determine the disease state (e.g., presence or severity of GI neoplasia) of a cat. In certain instances, the results of a statistical algorithm are compared to those results obtained for the same cat at an earlier time. In some aspects, the methods, systems, and code of the present invention can also be used to predict the progression of GI neoplasia, e.g., by determining a likelihood for GI neoplasia to progress either rapidly or slowly in a cat based on the presence or level of at least one marker in a sample. In other aspects, the methods, systems, and code of the present invention can also be used to predict the regression of GI neoplasia, e.g., by determining a likelihood for GI neoplasia to regress either rapidly or slowly in a cat based on the presence or level of at least one marker in a sample.

As used herein, the term “sensitivity” refers to the probability that a diagnostic method, system, or code of the present invention gives a positive result when the sample is positive, e.g., having chronic enteropathy or a clinical subtype thereof. Sensitivity is calculated as the number of true positive results divided by the sum of the true positives and false negatives. Sensitivity essentially is a measure of how well a method, system, or code of the present invention correctly identifies those with chronic enteropathy or a clinical subtype thereof from those without the disease. The statistical algorithms can be selected such that the sensitivity of classifying chronic enteropathy or a clinical subtype thereof or GI lymphoma is at least about 60%, and can be, for example, at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The term “specificity” refers to the probability that a diagnostic method, system, or code of the present invention gives a negative result when the sample is not positive, e.g., not having IBD or a clinical subtype thereof. Specificity is calculated as the number of true negative results divided by the sum of the true negatives and false positives. Specificity essentially is a measure of how well a method, system, or code of the present invention excludes those who do not have chronic enteropathy or a clinical subtype thereof from those who have the chronic enteropathy. The statistical algorithms can be selected such that the specificity of classifying chronic enteropathy or a clinical subtype thereof is at least about 50%, for example, at least about 50%, 55%, 60%, 65%, 70%, 75%, 80%, 85%, 86%, 87%, 88%, 89%, 90%, 91%, 92%, 93%, 94%, 95%, 96%, 97%, 98%, or 99%.

As used herein, the term “negative predictive value” or “NPV” refers to the probability that an individual identified as not having the disease or a clinical subtype thereof actually does not have the disease. Negative predictive value can be calculated as the number of true negatives divided by the sum of the true negatives and false negatives. Negative predictive value is determined by the characteristics of the diagnostic method, system, or code as well as the prevalence of the disease in the cat population analyzed. The statistical algorithms can be selected such that the negative predictive value in a population having a disease prevalence is in the range of about 50% to about 99% and can be, for example, at least about 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%.

The term “positive predictive value” or “PPV” refers to the probability that an individual identified as having chronic enteropathy or a clinical subtype thereof actually has the disease. Positive predictive value can be calculated as the number of true positives divided by the sum of the true positives and false positives. Positive predictive value is determined by the characteristics of the diagnostic method, system, or code as well as the prevalence of the disease in the cat population analyzed. The statistical algorithms can be selected such that the positive predictive value in a population having a disease prevalence is in the range of about 70% to about 99% and can be, for example, at least about 70%, 75%, 80%, 85%, 86%, 87%, 88%, 89%, 90%, 91%, 92%, 93%, 94%, 95%, 96%, 97%, 98%, or 99%.

Predictive values, including negative and positive predictive values, are influenced by the prevalence of the disease in the cat population analyzed. In the methods, systems, and code of the present invention, the statistical algorithms can be selected to produce a desired clinical parameter for a clinical population with a particular IBD or GI neoplasia prevalence. For example, learning statistical classifier systems can be selected for an IBD or GI neoplasia prevalence of up to about 1%, 2%, 3%, 4%, 5%, 6%, 7%, 8%, 9%, 10%, 15%, 20%, 25%, 30%, 35%, 40%, 45%, 50%, 55%, 60%, 65%, or 70%, which can be seen, e.g., in a veterinarian office.

As used herein, the term “overall agreement” or “overall accuracy” refers to the accuracy with which a method, system, or code of the present invention classifies a disease state. Overall accuracy is calculated as the sum of the true positives and true negatives divided by the total number of sample results and is affected by the prevalence of the disease in the cat population analyzed. For example, the statistical algorithms can be selected such that the overall accuracy in a patient population having a disease prevalence is at least about 60%, and can be, for example, at least about 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%.

The term “correlating” as used herein in reference to the use of biomarkers refers to comparing the presence or amount of the biomarker(s) in a cat to its presence or amount in cats known to suffer from, or known to be at risk of, a given condition; or in cats known to be free of a given condition. Often, this takes the form of comparing an assay result in the form of a biomarker concentration to a predetermined threshold selected to be indicative of the occurrence or nonoccurrence of a disease or the likelihood of some future outcome.

Population studies may also be used to select a decision threshold using Receiver Operating Characteristic (“ROC”) analysis to distinguish a diseased subpopulation from a non-diseased subpopulation. A false positive in this case occurs when the sample tests positive, but actually does not have the disease. A false negative, on the other hand, occurs when the sample tests negative, suggesting they are healthy, when they actually do have the disease. To draw a ROC curve, the true positive rate (TPR) and false positive rate (FPR) are determined. Since TPR is equivalent with sensitivity and FPR is equal to 1−specificity, the ROC graph is sometimes called the sensitivity vs (1−specificity) plot. A perfect test will have an area under the ROC curve of 1.0; a random test will have an area of 0.5. A threshold is selected to provide an acceptable level of specificity and sensitivity.

These measures include sensitivity and specificity, predictive values, likelihood ratios, diagnostic odds ratios, and ROC curve areas. The area under the curve (“AUC”) of a ROC plot is equal to the probability that a classifier will rank a randomly chosen positive instance higher than a randomly chosen negative one. The area under the ROC curve may be thought of as equivalent to the Mann-Whitney U test, which tests for the median difference between scores obtained in the two groups considered if the groups are of continuous data, or to the Wilcoxon test of ranks.

The term “statistical algorithm” or “statistical process” includes any of a variety of statistical analyses used to determine relationships between variables. In the present invention, the variables are the presence or level of at least one marker of interest. Any number of markers can be analyzed using a statistical algorithm described herein. For example, the presence or levels of 1, 2, 3, 4, 5, 6, 7, 8, 9, 10, 11, 12, 13, 14, 15, 16, 17, 18, 19, 20, 25, 30, 35, 40, 45, 50, or more markers can be included in a statistical algorithm. In one embodiment, logistic regression is used. In another embodiment, linear regression is used. In certain instances, the statistical algorithms of the present invention can use a quantile measurement of a particular marker within a given population as a variable. Quantiles are a set of “cut points” that divide a sample of data into groups containing (as far as possible) equal numbers of observations. For example, quartiles are values that divide a sample of data into four groups containing (as far as possible) equal numbers of observations. The lower quartile is the data value a quarter way up through the ordered data set; the upper quartile is the data value a quarter way down through the ordered data set. Quintiles are values that divide a sample of data into five groups containing (as far as possible) equal numbers of observations. The present invention can also include the use of percentile ranges of marker levels (e.g., textiles, quartile, quintiles, etc.), or their cumulative indices (e.g., quartile sums of marker levels, etc.) as variables in the algorithms (just as with continuous variables).

The statistical algorithms of the present invention comprise one or more learning statistical classifier systems. As used herein, the term “learning statistical classifier system” includes a machine learning algorithmic technique capable of adapting to complex data sets (e.g., panel of markers of interest) and making decisions based upon such data sets. In some embodiments, a single learning statistical classifier system such as a classification tree (e.g., random forest) is used. In other embodiments, a combination of 2, 3, 4, 5, 6, 7, 8, 9, 10, or more learning statistical classifier systems are used. Examples of learning statistical classifier systems include, but are not limited to, those using inductive learning (e.g., decision/classification trees such as random forests, classification and regression trees (C&RT), boosted trees, etc.), and genetic algorithms and evolutionary programming.

The learning statistical classifier systems described herein can be trained and tested using a cohort of samples (e.g., serological samples) from healthy, IBD and GI neoplasia cats. For example, samples from cats diagnosed by a veterinarian as having IBD or GI neoplasia during a biopsy and/or endoscopy are suitable for use in training and testing the learning statistical classifier systems of the present invention. Samples from healthy cats can include those that were not identified as IBD or GI neoplasia samples. One skilled in the art will know of additional techniques and diagnostic criteria for obtaining a cohort of cat samples that can be used in training and testing the learning statistical classifier systems of the present invention.

In certain instances, the methods of the invention are used in order to prognosticate the progression of IBD. The methods can be used to monitor the disease, both progression and regression. The term “monitoring the progression or regression of IBD” includes the use of the methods and marker profiles to determine the disease state (e.g., presence or severity of IBD) of a cat. In certain instances, the results of a statistical analysis are compared to those results obtained for the same cat at an earlier time. In some aspects, the methods, systems, and code of the present invention can also be used to predict the progression of IBD, e.g., by determining a likelihood for IBD to progress either rapidly or slowly in a cat based on the presence or level of at least one marker in a sample. In other aspects, the methods, systems, and code of the present invention can also be used to predict the regression of IBD, e.g., by determining a likelihood for IBD to regress either rapidly or slowly in an individual based on the presence or level of at least one marker in a sample.

In certain instances, the methods of the invention are used in order to prognosticate the progression of GI neoplasia. The methods can be used to monitor the disease, both progression and regression. The term “monitoring the progression or regression of GI neoplasia” includes the use of the methods and marker profiles to determine the disease state (e.g., presence or severity of GI neoplasia) of a pet. In certain instances, the results of a statistical analysis are compared to those results obtained for the same pet at an earlier time. In some aspects, the methods, systems, and code of the present invention can also be used to predict the progression of GI neoplasia, e.g., by determining a likelihood for GI neoplasia to progress either rapidly or slowly in a pet based on the presence or level of at least one marker in a sample. In other aspects, the methods, systems, and code of the present invention can also be used to predict the regression of GI neoplasia, e.g., by determining a likelihood for GI neoplasia to regress either rapidly or slowly in an individual based on the presence or level of at least one marker in a sample.

In certain instances, the methods of the invention are used in order to prognosticate the progression of an inflammatory condition. The methods can be used to monitor the disease, both progression and regression. The term “monitoring the progression or regression of inflammation” includes the use of the methods and marker profiles to determine the disease state (e.g., presence or severity of inflammation) of a patient. In certain instances, the results of a statistical analysis are compared to those results obtained for the same cat at an earlier time. In some aspects, the methods, systems, and code of the present invention can also be used to predict the progression of the symptoms, e.g., by determining a likelihood for the symptoms to progress either rapidly or slowly in the patient based on the presence or level of at least one marker in a sample. In other aspects, the methods, systems, and code of the present invention can also be used to predict the regression of IBD, e.g., by determining a likelihood for symptoms to regress either rapidly or slowly in the patient based on the presence or level of at least one marker in a sample.

In certain instances, the methods of the invention are used in order to prognosticate the progression of cancer. The methods can be used to monitor the disease, both progression and regression. The term “monitoring the progression or regression of cancer” includes the use of the methods and marker profiles to determine the disease state (e.g., presence or severity of cancer) of a patient. In certain instances, the results of a statistical analysis are compared to those results obtained for the same cat at an earlier time. In some aspects, the methods, systems, and code of the present invention can also be used to predict the progression of the symptoms, e.g., by determining a likelihood for the symptoms to progress either rapidly or slowly in the patient based on the presence or level of at least one marker in a sample. In other aspects, the methods, systems, and code of the present invention can also be used to predict the regression of IBD, e.g., by determining a likelihood for symptoms to regress either rapidly or slowly in the patient based on the presence or level of at least one marker in a sample.

### II. Diagnosing Chronic Enteropathies Including IBD and GI Neoplasia in Cats

In particular embodiments, the present invention provides methods and systems for detecting and measuring markers associated with IBD. Determining the presence and/or level of such markers is useful for accurately classifying whether a sample from a cat is associated with IBD or a clinical subtype or a clinically responsive subtype thereof. In some embodiments, the present invention is useful for classifying a sample from a cat as an IBD sample using empirical data (e.g., the presence or level of an IBD marker) and/or a statistical algorithm. The present invention is also useful for differentiating between different IBD sub-types using empirical data (e.g., the presence or level of an IBD marker) and/or a statistical algorithm. Accordingly, the present invention provides an accurate diagnostic prediction of IBD or a clinical subtype or a clinically responsive subtype thereof and prognostic information useful for guiding treatment decisions.

In particular embodiments, the present invention provides methods and systems for detecting and measuring markers associated with GI neoplasia. Determining the presence and/or level of such markers is useful for accurately classifying whether a sample from a cat is associated with GI neoplasia, such as lymphoma or a clinical subtype or a clinically responsive subtype thereof. In some embodiments, the present invention is useful for classifying a sample from a cat as a lymphoma sample using empirical data (e.g., the presence or level of a lymphoma marker) and/or a statistical algorithm. Accordingly, the present invention provides an accurate diagnostic prediction of lymphoma and prognostic information useful for guiding treatment decisions.

In a further aspect, the present invention provides a system for classifying whether a sample from a cat is associated with IBD and/or GI neoplasia, the system comprising: (a) a data acquisition module configured to produce a data set indicating the presence or level of at least one marker selected from the group consisting of an antimicrobial antibody, an inflammation antibody (e.g. integrin), an anti-structural antibody (e.g. keratin), an anti-proliferation antibody (e.g. Ki-67) and combinations thereof in the sample; (b) a data processing module configured to process the data set by applying a statistical process to the data set to produce a statistically derived decision classifying the sample as an IBD sample or Neoplasia sample or non-IBD sample based upon the presence or level of the at least one marker; and (c) a display module configured to display the statistically derived decision. In a related aspect, the present invention provides a system for classifying whether a sample from a cat is associated with a disease state, the system comprising: (a) a data acquisition module configured to produce a data set indicating the presence or level of at least one marker selected from the group consisting of an antimicrobial antibody, an anti-wound repair antibody, an antiinflammation antibody, an anti-proliferation antibody and combinations thereof in the sample; (b) a data processing module configured to process the data set by applying a statistical process to the data set to produce a statistically derived decision classifying the sample as IBD or GI neoplasia or non-IBD sample based upon the presence or level of the at least one marker; and (c) a display module configured to display the statistically derived decision. In one embodiment, the statistical process is a learning statistical classifier system. Examples of learning statistical classifier systems suitable for use in the present invention are described above. In certain instances, the statistical process is a single learning statistical classifier system. In certain other instances, the statistical process is a combination of at least two learning statistical classifier systems. In some instances, the data obtained from using the learning statistical classifier system or systems can be processed using a processing algorithm.

In other embodiments, the method of the present invention comprises determining the presence and/or level of anti-OmpC antibody (ACA), anti-Ki67 antibody (AKiA), anti-TK1 antibody, integrin antibody (AINTA), and anti-keratin (AKERA) in a sample such as serum, plasma, whole blood, or stool, and optionally additionally determining the presence or level of autoantibodies to polymorphonuclear leukocytes (PMN), autoantibody to inflammation markers, e.g., autoantibodies to calprotectin, lactoferritin, and/or C-reactive protein, and autoantibody to food sensitivity (anti-gliadin). A panel consisting of one or more of the IBD markers and neoplasia markers may be constructed and used for determining the presence or severity of IBD and/or neoplasia in the cat.

In some embodiments, the presence or level of at least two, three, four, five, six, seven, eight, nine, ten, or more IBD markers and neoplasia markers are determined in the cat's sample. In certain instances, the antimicrobial antibody comprises an anti-outer membrane protein C (ACA) antibody. In certain instances, the anti-outer membrane protein C antibody (ACA) comprises anti-OmpC immunoglobulin A (ACA-IgA). In certain other instances, the anti-inflammatory antibody comprises an integrin (AINTA) antibody. In certain instances, the anti-integrin antibody (AINTA) comprises anti-integrin immunoglobulin A (AINTA-IgA). In yet certain other instances, antibodies associated with proliferation associated with chronic enteropathies comprises an anti-Ki67 (AKiA) antibody. In certain instances, the anti-Ki67 antibody (AKiA) comprises anti-Ki67 immunoglobulin A (AKiA-IgA). In yet certain other instances, antibodies associated with proliferation associated with chronic enteropathies comprises an anti-TK1 antibody. In certain instances, the anti-TK1 antibody comprises anti-TK1 immunoglobulin A. In certain other instances, antibodies associated with wound repair associated with chronic enteropathies comprises an anti-keratin (AKERA) antibody. In certain instances, the anti-keratin antibody (AKERA) comprises anti-keratin immunoglobulin A (AGA-IgA).

The sample used for detecting or determining the presence or level of at least one marker is typically whole blood, plasma, serum, saliva, urine, stool (i.e., feces), tears, and any other bodily fluid, or a tissue sample (i.e., biopsy) such as a small intestine or colon sample. In some embodiments, the sample is serum, whole blood, plasma, stool, urine, or a tissue biopsy. In certain instances, the method of the present invention further comprises obtaining the sample from the cat prior to detecting or determining the presence or level of at least one marker in the sample.

In other embodiments, the method of the present invention comprises determining the presence or level of ACA, AKiA, AINTA, and/or AKERA in a sample such as serum, plasma, whole blood, or stool. A panel consisting of one or more of the IBD/GI neoplasia markers described above may be constructed and used for classifying the sample as an IBD sample or as a neoplasia sample.

In certain instances, the presence or level of at least one marker is determined using an immunoassay or an immunohistochemical assay. A non-limiting example of an immunoassay suitable for use in the method of the present invention includes an enzyme-linked immunosorbent assay (ELISA). Examples of immunohistochemical assays suitable for use in the method of the present invention include, but are not limited to, immunofluorescence assays such as direct fluorescent antibody assays, indirect fluorescent antibody (IFA) assays, anticomplement immunofluorescence assays, and avidin-biotin immunofluorescence assays. Other types of immunohistochemical assays include immunoperoxidase assays.

In some embodiments, the present invention is useful for classifying a sample from a cat as an IBD sample using a statistical algorithm (e.g., a learning statistical classifier system) and/or empirical data (e.g., the presence or level of an IBD marker; the presence or level of a neoplasia marker).

In certain embodiments, the method of the present invention further comprises sending the IBD and GI neoplasia classification results to a veterinarian. In another embodiment, the method of the present invention further provides a diagnosis in the form of a probability that the cat has IBD or a clinical subtype or a clinically responsive subtype thereof. For example, the patient can have about a 0%, 5%, 10%, 15%, 20%, 25%, 30%, 35%, 40%, 45%, 50%, 55%, 60%, 65%, 70%, 75%, 80%, 85%, 90%, 95%, or greater probability of having IBD or a clinical subtype thereof. In yet another embodiment, the method of the present invention further provides a prognosis of IBD in the cat. For example, the prognosis can be surgery, development of a clinical subtype of IBD, development of one or more symptoms, development of intestinal cancer, or recovery from the disease. In some instances, the method of classifying a sample as an IBD sample is further based on the symptoms (i.e., clinical factors) of the patient from which the sample is obtained. The symptoms or group of symptoms can be, for example, diarrhea, abdominal pain, cramping, fever, anemia, weight loss, anxiety, depression, and combinations thereof.

In another aspect, the present invention provides a method for monitoring the progression or regression of IBD and GI neoplasia in a cat, the method comprising: (a) determining the presence or level of at least one marker selected from the group consisting of antimicrobial antibody, antiinflammation autoantibody, anti-wound repair autoantibody, antiproliferation autoantibody, and combinations thereof in a sample from the cat; and (b) determining the presence or severity of IBD and GI neoplasia in the cat using a statistical algorithm based upon the presence or level of the at least one marker.

In yet another embodiment, the information obtained from the diagnostic test of the present invention may be used to monitor progression and/or treatment of IBD and neoplasia related diseases, including detection of residual disease after treatment of patients; to monitor onset of the conditions and progression and/or treatment; to modify therapeutic regimens, and to further optimize the selection of therapeutic agents. With this approach, therapeutic and/or diagnostic regimens can be individualized and tailored according to the data obtained at different times over the course of treatment, thereby providing a regimen that is individually appropriate.

In certain instances, the presence or level of at least one marker is determined using an immunoassay or an immunohistochemical assay. A non-limiting example of an immunoassay suitable for use in the method of the present invention includes an ELISA. Examples of immunohistochemical assays suitable for use in the method of the present invention include, but are not limited to, immunofluorescence assays such as direct fluorescent antibody assays, IFA assays, anticomplement immunofluorescence assays, and avidin-biotin immunofluorescence assays. Other types of immunohistochemical assays include immunoperoxidase assays.

In certain embodiments, the methods of the present invention can further comprise comparing the presence or severity of IBD or GI neoplasia to the presence or severity of IBD or GI neoplasia in the cat at an earlier time. As a non-limiting example, the presence or severity of IBD or GI neoplasia determined for a cat receiving a therapeutic agent useful for treating IBD and/or neoplasia can be compared to the presence or severity of IBD or GI neoplasia determined for the same cat before initiation of use of the therapeutic agent or at an earlier time in therapy. In certain other embodiments, the method can further comprise sending the IBD and/or GI neoplasia monitoring results to a veterinarian.

In yet another aspect, the present invention provides a computer-readable medium including code for controlling one or more processors to classify whether a sample from a cat is associated with IBD or GI neoplasia, the code including instructions to apply a statistical process to a data set indicating the presence or level of at least one marker selected from the group consisting of an antimicrobial antibody, an anti-wound repair antibody, an anti-inflammation antibody, an anti-proliferation antibody and combinations thereof in the sample to produce a statistically derived decision classifying the sample as an IBD sample or GI neoplasia sample or non-IBD sample based upon the presence or level of the at least one marker.

### III. Assays

Any of a variety of assays, techniques, and kits known in the art can be used to determine the presence or level of one or more markers in a sample to classify whether the sample is associated with IBD or GI neoplasia.

The present invention relies, in part, on determining the presence or level of at least one marker in a sample obtained from a cat. As used herein, the term “determining the presence of at least one marker” includes determining the presence of each marker of interest by using any quantitative or qualitative assay known to one of skill in the art. In certain instances, qualitative assays that determine the presence or absence of a particular trait, variable, or biochemical or serological substance (e.g., protein or antibody) are suitable for detecting each marker of interest. In certain other instances, quantitative assays that determine the presence or absence of RNA, protein, antibody, or activity are suitable for detecting each marker of interest. As used herein, the term “determining the level of at least one marker” includes determining the level of each marker of interest by using any direct or indirect quantitative assay known to one of skill in the art. In certain instances, quantitative assays that determine, for example, the relative or absolute amount of RNA, protein, antibody, or activity are suitable for determining the level of each marker of interest. One skilled in the art will appreciate that any assay useful for determining the level of a marker is also useful for determining the presence or absence of the marker.

A variety of immunoassay techniques, including competitive and non-competitive immunoassays (e.g., The immunoassay handbook 4^{th} edition, David Wild ed. Newnes, 2013) can be used to determine the presence or level of one or more markers in a sample. The term immunoassay encompasses techniques including, without limitation, enzyme immunoassays (EIA) such as enzyme multiplied immunoassay technique (EMIT), enzyme-linked immunosorbent assay (ELISA), direct ELISA, antigen capture ELISA, sandwich ELISA, IgM antibody capture ELISA (MAC ELISA), and microparticle enzyme immunoassay (META); capillary electrophoresis immunoassays (CEIA); radioimmunoassays (RIA); immunoradiometric assays (IRMA); fluorescence polarization immunoassays (FPIA); and chemiluminescence assays (CL). Liposome immunoassays, such as flow-injection liposome immunoassays and liposome immunosensors, are also suitable for use in the present invention. In addition, nephelometry assays, in which the formation of protein/antibody complexes results in increased light scatter that is converted to a peak rate signal as a function of the marker concentration, are suitable for use in the present invention. Nephelometry assays are commercially available from Beckman Coulter (Brea, CA) and can be performed using a Behring Nephelometer Analyzer.

The immunoassays described above are particularly useful for determining the presence or level of one or more markers in a sample. As a non-limiting example, an ELISA using OmpC protein or a fragment thereof is useful for determining whether a cat sample is positive for anti-OmpC antibodies, or for determining anti-OmpC antibody levels. An ELISA using calprotectin protein or a fragment thereof is useful for determining whether a cat sample is positive for anti-calprotectin antibodies, or for determining anti-calprotectin antibody levels. An ELISA using gliadin preparation derived from a plant extract or from a recombinant gene or a fragment thereof is useful for determining whether a cat sample is positive for gliadin antibodies, or for determining gliadin antibody levels. In particular, the immunoassays described above are useful for determining the presence or level of antimicrobial antibody, antiinflammation autoantibody, anti-wound repair autoantibody, antiproliferation autoantibody, and combinations thereof; e.g., for detecting the level of endogenous IgA antibodies to one or more, or all, of OmpC (ACA), Ki67 (AKiA), TK1, integrin (AINTA) and keratin (AKERA); in a cat, e.g., a cat having symptoms of chronic enteropathy.

Specific immunological binding of the antibody to the marker of interest can be detected directly or indirectly. Direct labels include fluorescent or luminescent tags, metals, dyes, radionuclides, and the like, attached to the antibody. An antibody labeled with iodine-125 (¹²⁵I) can be used for determining the levels of one or more markers in a sample. A chemiluminescence assay using a chemiluminescent antibody specific for the marker is suitable for sensitive, non-radioactive detection of marker levels. An antibody labeled with fluorochrome is also suitable for determining the levels of one or more markers in a sample. Examples of fluorochromes include, without limitation, DAPI, fluorescein, Hoechst 33258, R-phycocyanin, B-phycoerythrin, R-phycoerythrin, rhodamine, Texas red, and lissamine. Secondary antibodies linked to fluorochromes can be obtained commercially, e.g., goat F(ab′)2 anti-human IgG-FITC is available from Tago Immunologicals (Burlingame, CA).

Indirect labels include various enzymes well-known in the art, such as horseradish peroxidase (HRP), alkaline phosphatase (AP), β-galactosidase, urease, and the like. A horseradish-peroxidase detection system can be used, for example, with the chromogenic substrate tetramethylbenzidine (TMB), which yields a soluble product in the presence of hydrogen peroxide that is detectable at 450 nm. An alkaline phosphatase detection system can be used with the chromogenic substrate p-nitrophenyl phosphate, for example, which yields a soluble product readily detectable at 405 nm. Similarly, a β-galactosidase detection system can be used with the chromogenic substrate o-nitrophenyl-β-D-galactopyranoside (ONPG), which yields a soluble product detectable at 410 nm. A urease detection system can be used with a substrate such as urea-bromocresol purple (Sigma Immunochemicals; St. Louis, MO). A useful secondary antibody linked to an enzyme can be obtained from a number of commercial sources, e.g., goat anti-cat IgG-alkaline phosphatase can be purchased from Jackson ImmunoResearch (West Grove, PA.).

A signal from the direct or indirect label can be analyzed, for example, using a spectrophotometer to detect color from a chromogenic substrate; a radiation counter to detect radiation such as a gamma counter for detection of ¹²⁵I; or a fluorometer to detect fluorescence in the presence of light of a certain wavelength. For detection of enzyme-linked antibodies, a quantitative analysis of the amount of marker levels can be made using a spectrophotometer such as an EMAX Microplate Reader (Molecular Devices; Menlo Park, CA) in accordance with the manufacturer's instructions. If desired, the assays of the present invention can be automated or performed robotically, and the signal from multiple samples can be detected simultaneously.

The analysis of a plurality of markers may be carried out separately or simultaneously with one test sample. For separate or sequential assay of markers, suitable apparatuses include clinical laboratory analyzers such as the ElecSys (Roche), the AxSym (Abbott), the Access (Beckman), the AD VIA®, the CENTAUR® (Bayer), and the NICHOLS ADVANTAGE® (Nichols Institute) immunoassay systems. Particularly useful physical formats comprise surfaces having a plurality of discrete, addressable locations for the detection of a plurality of different markers. Such formats include protein microarrays, or “protein chips” and certain capillary devices (see, e.g., U.S. Pat. No. 6,019,944). In these embodiments, each discrete surface location may comprise antibodies to immobilize one or more markers for detection at each location. Surfaces may alternatively comprise one or more discrete particles (e.g., microparticles or nanoparticles) immobilized at discrete locations of a surface, where the microparticles comprise antibodies to immobilize one or more markers for detection.

As for the format of the test, it is understood that other diagnostic test devices may be adapted for the use of the present invention. For example, a strip test assay is well known in the art where the sample is applied to one end of the strip and the fluid migrates by capillary action up to the test zone. A sample can be any solution including body fluids (e.g. whole blood, serum or plasma, urine and the like).

Enzyme-linked immunosorbent assay (ELISA) methods are described above. For detection of the endogenous antibodies of the invention, for example, antigens to the endogenous antigens are attached to a surface. Then, the sample is contacted with the antigens, which act as bait to bind the endogenous antibodies, and a further specific antibody is applied over the surface, which can bind to the endogenous antibodies. This antibody is linked to an enzyme, and, in the final step, a substance containing the enzyme's substrate is added. The subsequent reaction produces a detectable signal, most commonly a color change in the substrate.

Several markers of interest may be combined into one test for efficient processing of a multiple of samples. In addition, one skilled in the art would recognize the value of testing multiple samples (e.g., at successive time points, etc.) from the same patient. Such testing of serial samples can allow the identification of changes in marker levels over time. Increases or decreases in marker levels, as well as the absence of change in marker levels, can also provide useful information to classify IBD and food sensitivity.

A panel consisting of one or more of the markers described above may be constructed to provide relevant information related to the approach of the present invention for classifying a cat sample as being associated with IBD and food sensitivity. Such a panel maybe constructed using 1, 2, 3, 4, 5, 6, 7, 8, 9, 10, 11, 12, 13, 14, 15, 16, 17, 18, 19, 20, 25, 30, 35, 40, or more individual markers. The analysis of a single marker or subsets of markers can also be carried out by one skilled in the art in various clinical settings.

The analysis of markers could be carried out in a variety of physical formats as well. For example, the use of microtiter plates or automation could be used to facilitate the processing of large numbers of test samples. Alternatively, single sample formats could be developed to facilitate treatment and diagnosis in a timely fashion.

In one aspect the invention relates to a kit for the detection of antibodies as described above, e.g., antimicrobial associated antibodies, inflammation-associated autoantibodies or wound repair-associated autoantibodies, proliferation-associated autoantibodies, IBD-associated antibodies or GI-neoplasia associated antibodies, in a sample comprising:
i. one or more peptide reagents as described above; and
ii. a means for detection of a complex formed between the peptide and antimicrobial associated antibodies, inflammation-associated autoantibodies or wound repair-associated autoantibodies, proliferation-associated autoantibodies, IBD-associated antibodies or GI-neoplasia associated antibodies.

The kit may contain ready to use reagents and the test results are advantageously obtained within several hours, e.g., less than six hours. For example, the kit may contain all ready to use reagents including coated plates, negative and positive controls, wash solution, sample diluent, conjugate, TMB and stop solutions. In some embodiments the solid phase of the test is coated with peptide antigen as described above. The peptide antigen can be chemically synthesized or expressed in E. coli or other suitable bacterial expression line. In the method and test kit any known and useful solid phase may be used. For example, MaxiSorp or PolySorp (Thermo Fisher Scientific) may be used and coated by applying a coating buffer which has a pH of, for example, 5, 7 or 9.5. The antigen is applied in a quantity of 0.1, 0.5, 1, 2, 3 or 10 μg/mL. A diluent may be used, for example (i) 0.14M NaCl, 2.7 mM KCl, Kathon 0.03%, Tween-20 0.1%; or (ii) 2% MgCl2, 6% Tween20 and 6% AO, 0.5% Casein sodium salt; or (iii) 25 mM Tris-HCl, 0.13 M, NaCl, 2.7 mM KCl, 1% Bovine Serum Albumin; or (iv) phosphate buffered saline, 1% Bovine Serum Albumin. The detection antibody is diluted, for example 1:2 000, or 1:5 000, or 1:20 000.

The method steps will be applied as required and may vary depending to the particular reagents applied. In a one embodiment the conditions and method steps are as follows:
a) Sample (1:100) in sample diluent (25 mM Tris-HCl, 0.13 M, NaCl, 2.7 mM KCl, 0.05% Tween-20, 1% Bovine Serum Albumin), 100 μl/well;
b) Incubate 1 hour, room temperature;
c) 3× wash (Tris buffered saline with 0.05% Tween20);
d) Conjugate ready-to-use, 100 μl/well;
e) Incubate 1 hour, room temperature;
f) 3× wash (Tris buffered saline with 0.05% Tween20);
g) TMB 100 μl 1/well, incubate 1 hour, room temperature;
h) Add stop solution (100 μl/well); and
i) Read out at 450 nm

In some embodiments the sample diluent contains casein sodium salt in a concentration of between 0.1 to 0.55%. For example, the sample diluent may contain 0.5% casein sodium salt and MgCl2, e.g., at a concentration of 2%.

In some embodiments the sample diluent contains bovine serum albumin in a concentration of between 0.1 to 5%.

In some embodiments the method of detection and/or the kit, is characterized by the inclusion of specific compounds, the use of particular dilutions of the capturing antigen and/or a particular amount and quality of capturing antigen coated onto the solid support used in the method of detection and the kit of the invention.

In some embodiments, the dilution of the antigen is chosen to be in the coating solution in a concentration of 0.25 to 5 μg/ml, for example, 0.5 to 1 μg/ml. The coating step is, for example, performed at pH 5 to 10, e.g. about 5, 7 or 9.5. The antigen as described in the specification and Examples, in some embodiments is used in amounts of 0.1, 0.5, 1, 2, or 4 μg/ml, e.g., 1 μg/ml.

In some embodiments, the method of detection and kit contains Tween, e.g. a Tween 20, or a comparable substance, e.g., a detergent with comparable characteristics. For example, the substance is contained in an amount of 0.05 to 0.5%, for example 0.1 to 0.2%.

In some embodiments the wash solution of the coating step contains NaCl 0.14M, KCl 2.7 mM; Kathon 0.03%, Tween20 0.1%, sample diluent comprises MgCl₂ 2%, aminoxid (AO) 6%, Tween20 6% and 0.5% casein. For example, the conjugate (where the patient is a cat, the anti-feline Ab conjugate) is used in a dilution of 1:2 000 to 1:30 000, e.g., 1:5 000 in a conjugate stabilizing buffer as a ready to use format.

The immunoassays described herein may be configured in a reagent impregnated test strip in which a specific binding assay is performed in a rapid and convenient manner with a minimum degree of skills and involvement.

For example, the disclosure provides embodiments including the following:
1. Methods for detecting endogenous IgA to Ki67 in serum obtained from a feline patient comprising
   a. contacting the serum with a feline Ki67 antigen, e.g., an antigen comprising the C-terminal portion of feline Ki67, e.g., according to embodiment 19 below; and
   b. detecting endogenous IgA to feline Ki67 using a labeled antibody which binds feline IgA.
2. Methods for detecting endogenous IgA to feline Ki67 in serum obtained from a feline patient using means (e.g., any of the means described herein) for separating endogenous IgA to feline Ki67 from serum and means (e.g. any of the means described herein) for detecting the endogenous IgA to feline Ki67 thus separated.
3. Methods for detecting endogenous IgA to TK1 in serum obtained from a feline patient comprising
   a. contacting the serum with a feline TK1 antigen, e.g., an antigen comprising the C-terminal portion of feline Ki67, e.g., an antigen according to embodiment 20 below; and
   b. detecting endogenous IgA to feline TK1 using a labeled antibody which binds feline IgA.
4. Methods for detecting endogenous IgA to feline TK1 in serum obtained from a feline patient using means (e.g., any of the means described herein) for separating endogenous IgA to feline TK1 from serum and means (e.g. any of the means described herein) for detecting the endogenous IgA to feline TK1 thus separated.
5. Methods for detecting endogenous IgA to OmpC from a feline microbiome in serum obtained from a feline patient comprising
   a. contacting the serum with an OmpC antigen, e.g., an antigen comprising an OmpC from E. coli from a feline intestine, e.g., according to embodiment 21 below; and
   b. detecting endogenous IgA to OmpC using a labeled antibody which binds feline IgA.
6. Methods for detecting endogenous IgA to OmpC from a feline microbiome in serum obtained from a feline patient using means (e.g. any of the means described herein) for separating endogenous IgA to OmpC from serum and means (e.g. any of the means described herein) for detecting the endogenous IgA to OmpC thus separated.
7. Methods for detecting endogenous IgA to feline integrin in serum obtained from a feline patient comprising
   a. contacting the serum with a feline integrin antigen, e.g., an antigen comprising the N-terminal portion of extracellular region of feline integrin beta subunit, e.g., an antigen according to embodiment 22 below; and
   b. detecting endogenous IgA to feline integrin using a labeled antibody which binds feline IgA.
8. Methods for detecting endogenous IgA to feline integrin in serum obtained from a feline patient using means (e.g. any of the means described herein) for separating endogenous IgA to feline integrin from serum and means (e.g. any of the means described herein) for detecting the endogenous IgA to feline integrin thus separated.
9. Methods for detecting endogenous IgA to feline keratin in serum obtained from a feline patient comprising
   a. contacting the serum with a feline keratin antigen, e.g., an antigen comprising feline keratin, type I cytoskeletal 18, e.g., an antigen according to embodiment 23, below; and
   b. detecting endogenous IgA to feline keratin using a labeled antibody which binds feline IgA.
10. Methods for detecting endogenous IgA to feline keratin in serum obtained from a feline patient using means (e.g. any of the means described herein) for separating endogenous IgA to feline keratin from serum and means (e.g. any of the means described herein) for detecting the endogenous IgA to feline keratin thus separated.
11. Methods for detecting endogenous IgA to a proliferation marker in serum obtained from a feline patient, e.g., wherein the proliferation marker is selected from Ki67 and TK1, the method comprising
   a. contacting the serum with an antigen for a proliferation marker, e.g., an antigen selected from
      a feline Ki67 antigen, e.g., an antigen comprising the C-terminal portion of feline Ki67, e.g., a Ki67 antigen as described in Embodiment 19; and
      a feline TK1 antigen, e.g., an antigen comprising the C-terminal portion of feline TK1, e.g., an antigen according to Embodiment 20; and
   b. detecting endogenous IgA to the proliferation marker using a labeled antibody which binds feline IgA.
12. A method of Embodiment 11 further comprising detecting endogenous IgA to a feline intestinal microbe in serum obtained from a feline patient, e.g., detecting endogenous IgA to OmpC in serum obtained from a feline patient, wherein the OmpC corresponds to an OmpC from a feline microbiome, comprising
   a. contacting the serum with an OmpC antigen, e.g., an antigen comprising an OmpC from E. coli from a feline intestine, e.g., an antigen according to embodiment 21; and
   b. detecting endogenous IgA to OmpC using a labeled antibody which binds feline IgA.
13. The method of Embodiment 11 or 12 further comprising a detecting endogenous IgA to a marker for wound repair or inflammation in serum obtained from a feline patient, e.g., detecting endogenous IgA to feline integrin or feline keratin in serum obtained from a feline patient, the method comprising
   a. contacting the serum with an antigenic portion of a marker for wound repair or inflammation, e.g., selected from a feline integrin antigen, e.g., an antigen comprising the N-terminal portion of extracellular region of feline integrin beta subunit, e.g., an antigen according to Embodiment 22; and an antigen comprising feline keratin, type I cytoskeletal 18, e.g., an antigen according to Embodiment 23; and
   b. detecting endogenous IgA to the marker for wound repair or inflammation using a labeled antibody which binds feline IgA.
14. A method of detecting combinations of endogenous IgA antibodies to (i) Ki67 (AKiA) and/or TK1; and (ii) one or more of OmpC (ACA), integrin (AINTA) and/or keratin (AKERA), in feline serum comprising
   a. contacting the serum with a feline Ki67 antigen, e.g., an antigen comprising the C-terminal portion of feline Ki67, e.g., an antigen according to claim 12; and detecting endogenous IgA to feline Ki67 using a labeled antibody which binds feline IgA, e.g., in accordance with the foregoing embodiments for detection of endogenous IgA to Ki67; and/or
      contacting the serum with a feline TK1 antigen, e.g., an antigen comprising the C-terminal portion of feline TK1, e.g., an antigen according to claim 12; and detecting endogenous IgA to feline TK1 using a labeled antibody which binds feline IgA, e.g., in accordance with claim 1; and
   b. contacting the serum with an OmpC antigen, e.g., an antigen comprising an OmpC from E. coli from a feline intestine, e.g., an antigen according to claim 13; and detecting endogenous IgA to OmpC using a labeled antibody which binds feline IgA, e.g., in accordance with the foregoing embodiments for detection of endogenous IgA to OmpC; and/or
      contacting the serum with a feline integrin antigen, e.g., an antigen comprising the N-terminal portion of extracellular region of feline integrin beta subunit, e.g., an antigen according to claim 14; and detecting endogenous IgA to feline integrin using a labeled antibody which binds feline IgA, e.g., in accordance with the foregoing embodiments for detection of endogenous IgA to feline integrin; and/or
      contacting the serum with a feline keratin antigen, e.g., an antigen comprising feline keratin, type I cytoskeletal 18, e.g., an antigen according to claim 15; and detecting endogenous IgA to feline keratin using a labeled antibody which binds feline IgA, e.g., in accordance with the foregoing embodiments for detection of endogenous IgA to feline keratin.
15. Methods for detecting combinations of endogenous IgA antibodies to OmpC (ACA), Ki67 (AKiA), integrin (AINTA) and/or keratin (AKERA), in feline serum comprising
   a. contacting the serum with a feline Ki67 antigen, e.g., an antigen comprising the C-terminal portion of feline Ki67; and detecting endogenous IgA to feline Ki67 using a labeled antibody which binds feline IgA, e.g., in accordance with embodiment 1; and/or
   b. contacting the serum with an OmpC antigen, e.g., an antigen comprising an OmpC from E. coli from a feline intestine; and detecting endogenous IgA to OmpC using a labeled antibody which binds feline IgA, e.g., in accordance with embodiment 3; and/or
   c. contacting the serum with a feline integrin antigen, e.g., an antigen comprising the N-terminal portion of extracellular region of feline integrin beta subunit; and detecting endogenous IgA to feline integrin using a labeled antibody which binds feline IgA, e.g., in accordance with embodiment 5; and/or
   d. contacting the serum with a feline keratin antigen, e.g., an antigen comprising feline keratin, type I cytoskeletal 18; and detecting endogenous IgA to feline keratin using a labeled antibody which binds feline IgA, e.g. in accordance with embodiment 7.
16. Any foregoing method, further comprising determining the presence or level of endogenous antibodies selected from one or more of endogenous antibodies to polymorphonuclear leukocytes (PMN), to calprotectin, to lactoferritin, to C-reactive protein, and to food sensitivity antigens (e.g., zein or gliadin); e.g. using methods as described in US20170248614A1, incorporated herein by reference.
17. Any foregoing method, comprising the steps of
   a. affixing an antigen (e.g., OmpC antigen, feline Ki67 antigen, feline integrin, and/or feline keratin antigen, e.g., an antigen according to any of embodiments 14-17) to a substrate,
   b. blocking any uncoated surfaces of the substrates with protein,
   c. exposing the antigens to the serum sample to allow formation of antigen-antibody complexes between the antigen and endogenous IgA,
   d. exposing the antigen-IgA complexes thus formed to the labeled antibody,
   e. detecting binding of the labeled antibody to the antigen-IgA complexes.
e.g., wherein the substrate is washed with buffer after each of steps a-d, e.g., wherein the labeled antibody is an anti-feline IgA antibody linked to an enzyme, e.g., comprising providing a substrate for the enzyme, and measuring the increase in optical density caused by the reaction of the enzyme with the substrate for the enzyme, wherein the increase in optical density correlates with the presence and amount of endogenous IgA bound to antigen, e.g., wherein the enzyme is horseradish peroxidase (HRP) and the substrate is 3,3′,5,5′-Tetramethylbenzidine (TMB).
18. A feline Ki67 antigen comprising feline Ki67 or an antigenic fragment thereof, e.g., the C-terminal portion of feline Ki67, e.g., comprising SEQ ID NO: 4, covalently linked to a label and/or one or more fusion tags, e.g., selected from a polyhistidine tag (e.g., 6×His tag), a Small Ubiquitin-like Modifier (SUMO) tag, and a glutathione 5-transferase (GST) tag; e.g., a feline Ki67 antigen which is a fusion protein comprising SEQ ID NO: 4 and comprising any one or more of SEQ ID NO. 13-17, e.g., a feline Ki67 antigen of SEQ ID NO: 5 or SEQ ID NO: 6.
19. A feline TK1 antigen comprising feline TK1 or an antigenic fragment thereof, e.g., the C-terminal portion of feline TK1, e.g., comprising SEQ ID NO: 18, covalently linked to a label and/or one or more fusion tags, e.g., selected from a polyhistidine tag (e.g., 6×His tag), a Small Ubiquitin-like Modifier (SUMO) tag, and a glutathione 5-transferase (GST) tag; e.g., a feline TK1 antigen which is a fusion protein comprising SEQ ID NO: 18 and further comprising any one or more of SEQ ID NO. 13-17, e.g., a feline TK1 antigen comprising SEQ ID NOS: 19, 20, 21, or 22.
20. An OmpC antigen comprising an OmpC corresponding to OmpC from E. coli from a feline intestine, e.g. comprising SEQ ID NO: 1 covalently linked to a label and/or one or more fusion tags, e.g., selected from a polyhistidine tag (e.g., 6×His tag), a Small Ubiquitin-like Modifier (SUMO) tag, and a glutathione 5-transferase (GST) tag; e.g., an OmpC antigen which is a fusion protein comprising SEQ ID NO: 1 and further comprising any one or more of SEQ ID NO. 13-17, e.g., an OmpC antigen of SEQ ID NO: 2 or SEQ ID NO: 3.
21. A feline integrin antigen comprising a feline integrin or antigenic fragment thereof, e.g. comprising the N-terminal portion of extracellular region of feline integrin beta subunit, e.g., comprising SEQ ID NO: 7, covalently linked to a label and/or one or more fusion tags, e.g., selected from a polyhistidine tag (e.g., 6×His tag), a Small Ubiquitin-like Modifier (SUMO) tag, and a glutathione 5-transferase (GST) tag; e.g., a feline integrin antigen which is a fusion protein comprising SEQ ID NO: 7 and comprising any one or more of SEQ ID NO. 13-17, e.g., a feline integrin antigen of SEQ ID NO: 8 or SEQ ID NO: 9.
22. A feline keratin antigen comprising feline keratin or antigenic fragment thereof, e,g. comprising feline keratin, type I cytoskeletal 18, e.g. comprising SEQ ID NO: 10, covalently linked to a label and/or one or more fusion tags, e.g., selected from a polyhistidine tag (e.g., 6×His tag), a Small Ubiquitin-like Modifier (SUMO) tag, and a glutathione 5-transferase (GST) tag; e.g., a feline keratin antigen which is a fusion protein comprising SEQ ID NO: 10 and comprising any one or more of SEQ ID NO. 13-17, e.g., a feline keratin antigen of SEQ ID NO: 11 or SEQ ID NO: 12.
23. A gene comprising a DNA sequence encoding an antigen according to any of embodiments 14-18, e.g. encoding a sequence according to any of SEQ ID NOS: 1-12 or 18-22, operably linked to a heterologous promoter.
24. Methods of treating a feline having serum levels of greater than 75, e.g., greater than 90, e.g. 75-250, ELISA units per milliliter of endogenous IgA antibodies to one or more, or all, of OmpC (ACA), Ki67 (AKiA), TK1, integrin (AINTA) and keratin (AKERA), e.g. as detected pursuant to any foregoing method of detection, e.g., wherein the feline has a neoplasia, e.g., lymphoma, comprising administering to the feline an effective amount of a chemotherapeutic agent.
25. Methods of treating a feline having serum levels of 25-75, e.g. 30-60, ELISA units per milliliter of endogenous IgA antibodies to one or more or all of OmpC (ACA), Ki67 (AKiA), TK1, integrin (AINTA) and keratin (AKERA), e.g. as detected pursuant to any foregoing method of detection, e.g., wherein the feline is suffering from an inflammatory condition, e.g., inflammatory bowel disease, comprising administering to the feline an effective amount of an anti-inflammatory agent, e.g. a corticosteroid, e.g., a corticosteroid selected from prednisone, prednisolone, dexamethasone, triamcinolone, budesonide, and methylprednisolone, e.g., selected from prednisone and budesonide.
26. Methods of diagnosing and differentiating between neoplasia, e.g., gastrointestinal neoplasia, and an inflammatory condition, e.g., inflammatory bowel disease, in a feline exhibiting symptoms of gastrointestinal disorder, comprising
   a. detecting the level of endogenous IgA antibodies to one or more, or all, of OmpC (ACA), Ki67 (AKiA), TK1, integrin (AINTA) and keratin (AKERA), e.g., in accordance with any of the foregoing methods of detecting endogenous antibodies; and
   b. diagnosing neoplasia, e.g., gastrointestinal neoplasia, when the feline exhibits serum levels of greater than 75, e.g., greater than 90, e.g. 75-250, ELISA units per milliliter of endogenous IgA antibodies to one or more, or all, of OmpC (ACA), Ki67 (AKiA), TK1, integrin (AINTA) and keratin (AKERA),
   c. diagnosing an inflammatory condition, e.g. inflammatory bowel disease, when the feline exhibits serum levels of 25-75, e.g. 30-60, ELISA units per milliliter of endogenous IgA antibodies to one or more or all of OmpC (ACA), Ki67 (AKiA), TK1, integrin (AINTA) and keratin (AKERA).
27. A method of monitoring progression and/or treatment of a chronic enteropathy in a feline, e.g., an inflammatory condition, e.g. IBD, and/or a neoplasia related disease, e.g., gastrointestinal lymphoma, including detection of residual disease after treatment of patients; e.g., over a period of time, e.g., before and after a course of treatment; comprising: (a) determining the presence or level of at least one marker selected from the group consisting of antimicrobial antibody, antiinflammation autoantibody, anti-wound repair autoantibody, antiproliferation autoantibody, and combinations thereof [e.g., detecting the level of endogenous IgA antibodies to one or more, or all, of OmpC (ACA), Ki67 (AKiA), TK1, integrin (AINTA) and keratin (AKERA), e.g., in accordance with any of the foregoing methods of detecting endogenous antibodies] in a sample from the cat; and (b) determining the presence or severity of IBD and GI neoplasia in the cat using a statistical algorithm based upon the presence or level of the at least one marker, e.g., wherein the presence, absence, and/or level of the endogenous antibodies correlates with the progression or remission of the chronic enteropathy; e.g., including methods of monitoring onset of the conditions and progression and/or treatment; methods of identifying a need to modify therapeutic regimens, and/or to optimize the selection of therapeutic agents; e.g., in conjunction with a method of treating a chronic enteropathy in a feline, e.g., in accordance with Embodiment 20 or 21 above.
28. Kits, e.g. for use in accordance with any of the above methods for detecting endogenous antibodies, comprising
   a. One or more antigens as described above comprising antigenic portions of OmpC, feline Ki67, feline TK1, feline integrin, and/or feline keratin, e.g., one or more antigens comprising any of SEQ ID NOS 1-12 or 18-22; and
   b. a labeled antibody which binds feline IgA.

Other features and advantages of the invention are apparent from the following description of the embodiments thereof, and from the claims.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

### EXAMPLES

The following examples are offered to illustrate, but not to limit, the claimed invention in any manner.

### Example 1—Identification of Microorganisms Isolated from Biopsy Samples from Cats Presenting Chronic Enteropathies

This example illustrates the identification of microorganism isolated from samples from cats with chronic enteropathies.

Microorganism cultures are isolated from biopsy samples from cats with chronic enteropathies after obtaining informed consent from owners and genotyped using 1.6S rRNA gene sequencing.

The following organisms including the genus and the species are isolated from biopsy samples from 6 cats with chronic enteropathies:
(i) Gram negative: Escherichia (Escherichia coli) and Pseudonomas (Pseudonomas lundenis/taetrolens, Pseudomonas mucidolens/synxantha, Pseudomonas fulva);
(ii) Gram positive: Staphylococcus (Staphylococcus felis), Rhodococcus (Rhodococcus erythropolis), Streptococcus (Streptococcus bovis).

### Example 2—Cloning of Markers

This example illustrates (i) the cloning and the preparation of recombinant markers OmpC, feline Ki67, feline TK1, feline integrin, and feline keratin18; and (ii) the analysis of anti-OmpC antibody (ACA), anti-Ki67 antibody (AKiA), anti-TK1 antibody, integrin antibody (AINTA), and anti-keratin (AKERA) levels in a sample using an ELISA assay.

The full coding regions or fragment thereof are subcloned into an expression vector with the N-terminal region of the coding gene operably linked to a start codon and an inducible promoter system. The nucleic acid sequence of the coding region is designed to include a polyhistidine tag to create a HIS-fusion polypeptide. After expression in E. coli, the fusion polypeptides are purified using a nickel purification column using standard expression and purification technique. The recombinant polypeptides for OmpC, Ki67, integrin, keratin and TK1 utilized for the preparation of the coating material is selected from sequences SEQ ID No: 1 to 12 and 19-22.

Feline IgA antibodies and autoantibodies against specific antigens are detected by ELISA in serum samples collected from cats and stored for short period of time at 2 to 8° C. or −10 to −20° C. and for long period of time at −50 to −80° C. until analysis.

Sera from healthy and diseased cats are analyzed in duplicate for IgA reactivity to OmpC and feline Ki67, feline integrin and feline keratin as follows. Microtiter plates are coated with 100 μL/well at 0.2-0.5 μg/mL antigen in carbonate solution (100.0 mM NaHCO₃—Na2CO3 buffer, pH 9.5±0.5). The plates are washed thrice with saline based solution (pH 7.4±0.2) and blocked with 200 μL/well the same solution containing 1% BSA for 1-hour. After washing the plates thrice, the standard and sample sera are added to each well. After 1-hour incubation, the plates are washed thrice and then incubated with horseradish peroxidase (HRP)-anti-cat IgA antibody. After a wash step, the plates are developed using 100 μL/well of 3,30,5,50-tetramethylbenzidine (TMB) substrate. The reaction is stopped with H2SO4 and the OD is measured at 450 nm using an ELISA plate reader.

### Example 3—Determination of ACA, AKiA, AINTA, and AKERA Levels in Cat Serum Samples

This example illustrates an analysis of anti-OmpC antibody (ACA), anti-Ki-67 antibody (AKiA), integrin antibody (AINTA), and anti-keratin (AKERA) using an ELISA assay in serum samples. Serum samples are collected from three cohorts of cats: (i) the “IBD” cohort includes cats confirmed with the diagnosis of IBD based on the chronicity of gastrointestinal signs and the histological inflammatory findings; (ii) the “Lymphoma” cohort includes cats confirmed with the diagnosis of GI Lymphoma based on the clinical signs and the histological findings; and (iii) the “Normal” cohort including cats with no apparent gastrointestinal symptoms at the time of sample collection.

### Study Design and Inclusion Criteria.

This is a multicenter study designed to develop methods and systems to accurately detect and measure the presence and/or levels of endogenous antibodies to markers associated with chronic enteropathies, mainly inflammatory bowel disease (IBD) and gastrointestinal neoplasia in cats. Such methods and systems identify whether a sample from the patient is associated with IBD or GI neoplasia, by using non-invasive means, thus conveniently providing information useful for guiding treatment decisions.

Client-owned cats of any sex or breed, older than 1 year of age and presenting clinical signs of IBD and GI neoplasia are included in a prospective clinical study after obtaining owner's written consent. Inclusion criteria include a history of vomiting, diarrhea, anorexia, weight loss, or any combination of these clinical signs occurring on a continuous or intermittent basis over a period of at least three weeks. Cats with a history of recurrence following treatment or chronic antibiotic responsive diarrhea are also included as well as cats exhibiting positive standard diagnosis for GI neoplasia including GI lymphoma confirmed by any recognized diagnostic test. Cats with double diagnosis of IBD and GI neoplasia are also enrolled. Furthermore, a complete clinical evaluation is performed to exclude subjects with concurrent parasitism, toxin ingestion, and any other significant GI conditions determined by standard commercially available tests.

IBD and GI neoplasia (including lymphoma) diagnosis is confirmed by abdominal ultrasound, histologic evaluation and examination of full-thickness biopsy specimens obtained from ≥2 small bowel sites during laparotomy. Slides are histologically graded by board-certified veterinary pathologists, following the World Small Animal Veterinary Association (WSAVA) International Gastrointestinal (GI) guidelines. Multiple morphological parameters (i.e. epithelial injury, crypt distension, lacteal dilatation, mucosal fibrosis) and inflammatory histological parameters (such as plasma cells, lamina propria lymphocyte, eosinophils and neutrophils) are scored, and the resulting final scores are subdivided into histological severity groups: WSAVA score of 0=normal, 1-6=mild, 7-12=moderate, >13=severe.

The study also includes a cohort of apparently healthy cats (Normal) of various ages, sex, and breeds presenting no significant GI clinical signs.

### Statistical Analysis

Statistical analysis is conducted using the Graphpad Prism (GraphPad Software, La Jolla California USA) or Microsoft Office Excel (2013, Microsoft, Redmond, WA, USA). Mean, median, minimum, maximum, and percentile are calculated. Data are analyzed by the Kruskal-Wallis test. Statistical analyses include area under receiver operating characteristic (ROC) curves and calculations of diagnostic sensitivity and specificity as appropriate for each of the markers (univariate analysis) and for a combination of markers (multivariate analysis). Measures of performance, sensitivity and specificity, may be computed using multiple reference values. A p-value <0.05 is considered significant.

### Results.

The IBD cohort, the GI neoplasia cohort, and the Normal cohort include 25, 25, and 56 cats respectively, of various ages, gender and breeds presenting with chronic enteropathy symptoms. Levels of IgA antibodies to OmpC (ACA), Ki67 (AKiA), integrin (AINTA) and keratin (AKERA) are determined in all enrolled subjects by the ELISA method developed in the present invention. Antibody levels are determined relative to a standard/calibrator/reference using the Softmax software (Molecular Devices) and are expressed as ELISA units (EU/mL). The recombinant polypeptides for OmpC, Ki67, integrin, and keratin utilized for the preparation of the coating material derived from sequences SEQ ID No: 3, SEQ ID No: 6, SEQ ID No: 9 and SEQ ID No: 12, respectively.

Typical results obtained with serum samples from IBD Cats, GI neoplasia Cats, and Normal Cats using the ELISA method described above are reported herein.

Data are compared using the Kruskal-Wallis test and are expressed as Mean±Standard Error of the Mean (SEM) using ELISA units (EU/mL). The data show that there is a significant statistical difference between the Normal cohort vs the IBD cohort vs the Lymphoma. The differentiation between cohorts as well as the statistical significance is further assessed by quantifying the area under the curve (AUC) from the receiver operating characteristic (ROC) curve. The results demonstrate that the markers are able to differentiate between cohorts in all cases with p values <0.0001. From these data, sera with circulating ACA, AKiA, AINTA, and AKERA antibodies may be categorized as non-IBD, IBD, or GI neoplasia. These three categories are defined by analysis of area under receiver operating characteristic (ROC) curves and calculations of diagnostic sensitivity and specificity as appropriate for each of the markers (univariate analysis) and for a combination of markers (multivariate analysis).

Overall, these results indicate that the method of detecting in a sample the presence and/or level of endogenous antibodies to OmpC, Ki67, integrin, and keratin markers associated with inflammatory bowel disease (MD) and GI neoplasia, can be utilized to evaluate and differentiate IBD and GI neoplasia in cats.

TABLE 1
Mean ± SEM results obtained for four markers, OmpC
(ACA), Ki67 (AKiA), integrin (AINTA) and keratin (AKERA)
for the IBD, neoplasia, and Normal feline cohorts.

| | | GI Neoplasia | | |
|---|---|---|---|---|
| Markers | IBD Cats | Cats | Normal Cats | p values |
| ACA | 35.30 ± 6.20 | 165.8 ± 32.55 | 9.713 ± 0.98 | <0.0001 |
| AKiA | 33.82 ± 4.92 | 154.6 ± 23.81 | 11.21 ± 1.21 | <0.0001 |
| AINTA | 29.41 ± 4.16 | 151.4 ± 26.50 | 7.342 ± 0.81 | <0.0001 |
| AKERA | 35.80 ± 5.34 | 193.7 ± 42.46 | 10.74 ± 1.16 | <0.0001 |

TABLE 2
Area under the curve values (AUC) obtained for ROC curves using
OmpC (ACA), Ki67 (AKiA), integrin (AINTA) and keratin (AKERA)
markers for discrimination between the Normal vs. IBD, Normal
vs. Neoplasia, and IBD vs. Neoplasia feline cohorts.

| | Normal | Normal vs | IBD vs |
|---|---|---|---|
| AUC | vs IBD | Neoplasia | Neoplasia |
| ACA | 0.9064 | 0.9993 | 0.9488 |
| AKiA | 0.8714 | 0.9993 | 0.9600 |
| AINTA | 0.9200 | 0.9986 | 0.9584 |
| AKERA | 0.8814 | 0.9979 | 0.9552 |

TABLE 3
Results obtained for four markers, OmpC (ACA), Ki67 (AKiA), integrin (AINTA) and
keratin (AKERA) for serum samples collected from cats with various types of cancer of the GI
tract. Characterization of cancer was performed by histopathology of full-thickness biopsy
specimen. Values are expressed as EU/mL.

| | | | | | | | Avg- 4 |
|---|---|---|---|---|---|---|---|
| Sample | Cancer | Description | ACA | AKiA | AINTA | AKERA | markers |
| 1 | Small cell, | Mucosal villous propria of | 157.56 | 129.10 | 102.71 | 114.54 | 125.98 |
| | low-grade | jejunum is expanded by an | | | | | |
| | lymphoma | infiltrate of small uniform | | | | | |
| | | lymphoid cells with scant | | | | | |
| | | cytoplasm and round hyper- | | | | | |
| | | chromatic nuclei. Mitoses | | | | | |
| | | are rare. Lymphocytic | | | | | |
| | | epitheliotropism is seen with | | | | | |
| | | formation of few intraepithelial | | | | | |
| | | microabscesses, primarily | | | | | |
| | | in glandular epithelium. | | | | | |
| 2 | High-grade, | The stomach lamina propria is | 60.86 | 61.69 | 66.39 | 61.35 | 62.57 |
| | large-cell | infiltrated by neoplastic round | | | | | |
| | lymphoma | cells forming dense sheets | | | | | |
| | | with moderate anisocytosis | | | | | |
| | | and anisokaryosis that extends | | | | | |
| | | into and effaces the intestinal | | | | | |
| | | wall. Cells are large, have | | | | | |
| | | indistinct cell borders, small | | | | | |
| | | amounts of eosinophilic | | | | | |
| | | cytoplasm, round nuclei, | | | | | |
| | | stippled chromatin and 0-1 | | | | | |
| | | nucleoli. Mitoses average 3 | | | | | |
| | | per HPF (2.37 mm2). | | | | | |
| 3 | Adeno- | Neoplastic mass in small | 576.64 | 559.63 | 473.17 | 897.73 | 626.79 |
| | carcinoma | intestine jejunum consisting | | | | | |
| | | of nests and tubules extending | | | | | |
| | | from the mucosal epithelium | | | | | |
| | | to mesentery and infiltrates | | | | | |
| | | the muscular layers. Neoplastic | | | | | |
| | | cells with moderate | | | | | |
| | | anisokaryosis and anisocytosis | | | | | |
| | | have indistinct borders, | | | | | |
| | | small amounts of eosinophilic | | | | | |
| | | to mucinous cytoplasm, oval | | | | | |
| | | nuclei. There are 4 mitoses | | | | | |
| | | in 10 HPF (2.37 mm2). | | | | | |
| 4 | Mast cell | The ileum mucosa and sub- | 67.03 | 74.74 | 67.30 | 79.26 | 72.08 |
| | tumor | mucosa are affected by a | | | | | |
| | | mass consisting of coalescing | | | | | |
| | | nodules of neoplastic cells | | | | | |
| | | with mild anisokaryosis | | | | | |
| | | and anisocytosis. No | | | | | |
| | | mitoses are seen in 10 HPF | | | | | |
| | | (2.37 mm2). Small number | | | | | |
| | | of lymphocytes and pus cells | | | | | |
| | | infiltrate the mucosa. | | | | | |
| 5 | Exocrine | Neoplasm in pancreas is | 142.95 | 167.75 | 146.46 | 191.82 | 162.24 |
| | adeno- | composed of nests of | | | | | |
| | carcinoma | neoplastic cells with poorly | | | | | |
| | | distinct cell borders, | | | | | |
| | | moderate amounts of | | | | | |
| | | eosinophilic cytoplasm, oval | | | | | |
| | | nuclei, coarsely granular | | | | | |
| | | chromatin. There are 4 | | | | | |

mitoses in 10 HPF (2.37
mm2).

### Example 4—Determination of ACA, AKiA, AINT, and AKER Levels in Cat Serum Samples in a Longitudinal Study

This example illustrates an analysis of anti-OmpC antibody level (ACA), anti-Ki67 antibody level (AKiA), anti-integrin antibody level (AINTA), and anti-keratin antibody level (AKERA) using cat serum samples to monitor the marker levels during the evolution of the disease.

In this study, serum samples are collected from cats with chronic enteropathy symptoms such as vomiting, diarrhea, anorexia, weight loss, or some combination over a long period of time. Serum samples are collected at the initial visit and at a follow-up visit after completion of treatment prescribed by the attending clinician. Evidence of inflammatory bowel disease or GI neoplasia (including lymphoma) is confirmed by a pathologist based on a biopsy.

Levels of feline IgA antibodies to OmpC (ACA), Ki67 (AKiA), integrin (AINTA) and keratin (AKERA) are determined in all enrolled subjects by the ELISA method developed in the present invention.

Typical results are listed for cats. These results indicate that the method of detecting the presence and/or level of one or more endogenous antibodies associated with inflammatory bowel disease (IBD) and lymphoma in a sample can be utilized to detect and monitor IBD and lymphoma. The longitudinal samples appear to detect improvements in outcome measured over time reflecting overall response to treatment. This study is building support for the test to be helpful at identifying cats which remain in remission or which may need further treatment.

TABLE 4
Results of levels expressed in ELISA units per milliliter (EU/mL) of four markers OmpC
(ACA), Ki67 (AKiA), integrin (AINTA) and keratin (AKERA) in feline serum samples collected
prior and after treatment from cats diagnosed with GI neoplasia or IBD exhibiting chronic
enteropathy symptoms.

| Sample | Diagnosis | Visits | Symptoms | Treatment | ACA | AKiA | AINTA | AKERA | Average | % Baseline |
|---|---|---|---|---|---|---|---|---|---|---|
| Cat-1 | Lymphoma | Visit 1 | CE | P, C | 88.7 | 108.5 | 109.5 | 96.5 | 100.8 | 100.0 |
| | | FU | None | | 13.8 | 17.6 | 12.6 | 13.6 | 14.4 | 14.3 |
| Cat-2 | Lymphoma | Visit 1 | CE | P, C, B1 | 130.7 | 142.0 | 188.0 | 169.6 | 157.6 | 100.0 |
| | | FU | Improved | | 92.0 | 102.2 | 109.6 | 119.3 | 105.8 | 67.1 |
| Cat-3 | Lymphoma | Visit 1 | CE | P, C | 268.5 | 282.9 | 270.3 | 354.8 | 294.1 | 100.0 |
| | | FU | None | | 63.4 | 55.5 | 31.5 | 38.6 | 47.3 | 16.1 |
| Cat-4 | Lymphoma | Visit 1 | CE | D | 109.2 | 93.8 | 92.0 | 107.3 | 100.6 | 100.0 |
| | | FU | Quiet | | 155.2 | 141.8 | 139.7 | 166.5 | 150.8 | 149.9 |
| Cat-5 | IBD | Visit 1 | CE | D, A | 48.8 | 38.3 | 43.7 | 51.1 | 45.5 | 100.0 |
| | | | Some | | | | | | | |
| | | FU | improv. | | 41.3 | 45.2 | 25.7 | 27.3 | 34.9 | 76.7 |
| Cat-6 | IBD | Visit 1 | CE | Bu | 51.0 | 47.7 | 51.2 | 50.9 | 50.2 | 100.0 |
| | | FU | Rare | | 0.1 | 3.3 | 19.9 | 33.8 | 29.3 | 58.3 |
| Cat-7 | IBD | Visit 1 | CE | P | 30.8 | 27.7 | 28.9 | 37.5 | 31.2 | 100.0 |
| | | | No | | | | | | | |
| | | FU | improv. | | 28.8 | 34.3 | 19.2 | 37.4 | 29.9 | 95.8 |

CE: Chronic enteropathy symptoms;
FU: Follow-up visit;
Treatment:
A: Antibiotics,
B: Budesonide,
B1: Vitamin B12,
C: Chemotherapy,
D: Diet,
P: Prednisone

SEQUENCE LISTING

| SEQ ID | Description | Amino Acid Sequence |
|---|---|---|
| SEQ ID NO: 1 | OmpC isolated from E. coli | AEVYNKDGNKLDLYGKVDGLHYFSDDKSVDGDQTYMRLGF |
| | collected from cat biopsy | KGETQVTDQLTGYGQWEYQIQGNSAENENNSWTRVAFAGL |
| | | KFQDVGSFDYGRNYGVVYDVTSWTDVLPEFGGDTYGSDNF |
| | | MQQRGNGFATYRNTDFFGLVDGLNFAVQYQGKNGSVDGEG |
| | | MTNNGRGALRQNGDGVGGSITYDYEGFGIGAAVSSSKRTD |
| | | AQNGTYVVDNVTHNYIGTGDRAETYTGGLKYDANNIYLAA |
| | | QYTQTYNATRVGSLGWANKAQNFEAVAQYQFDFGLRPSLA |
| | | YLQSKGKNLGVINGRNYDDEDILKYVDVGATYYFNKNMST |
| | | YVDYKINLLDDNQFTRDAGINTDNIVALGLVYQF |
| SEQ ID NO: 2 | OmpC with a fusion tag | MGSSHHHHHHAEVYNKDGNKLDLYGKVDGLHYFSDDKSVD |
| | comprising a hexa-His | GDQTYMRLGFKGETQVTDQLTGYGQWEYQIQGNSAENENN |
| | sequence | SWTRVAFAGLKFQDVGSFDYGRNYGVVYDVTSWTDVLPEF |
| | | GGDTYGSDNFMQQRGNGFATYRNTDFFGLVDGLNFAVQYQ |
| | | GKNGSVDGEGMTNNGRGALRQNGDGVGGSITYDYEGFGIG |
| | | AAVSSSKRTDAQNGTYVVDNVTHNYIGTGDRAETYTGGLK |
| | | YDANNIYLAAQYTQTYNATRVGSLGWANKAQNFEAVAQYQ |
| | | FDFGLRPSLAYLQSKGKNLGVINGRNYDDEDILKYVDVGA |
| | | TYYFNKNMSTYVDYKINLLDDNQFTRDAGINTDNIVALGL |
| | | VYQF |
| SEQ ID NO: 3 | OmpC with a fusion tag | MGSSHHHHHHGSGLVPRGSASMSDSEVNQEAKPEVKPEVK |
| | comprising a hexa-His | PETHINLKVSDGSSEIFFKIKKTTPLRRLMEAFAKRQGKE |
| | sequence, a thrombin | MDSLRFLYDGIRIQADQTPEDLDMEDNDIIEAHREQIGGR |
| | cleavage sequence, and a | AEVYNKDGNKLDLYGKVDGLHYFSDDKSVDGDQTYMRLGF |
| | SUMO sequence. | KGETQVTDQLTGYGQWEYQIQGNSAENENNSWTRVAFAGL |
| | | KFQDVGSFDYGRNYGVVYDVTSWTDVLPEFGGDTYGSDNF |
| | | MQQRGNGFATYRNTDFFGLVDGLNFAVQYQGKNGSVDGEG |
| | | MTNNGRGALRQNGDGVGGSITYDYEGFGIGAAVSSSKRTD |
| | | AQNGTYVVDNVTHNYIGTGDRAETYTGGLKYDANNIYLAA |
| | | QYTQTYNATRVGSLGWANKAQNFEAVAQYQFDFGLRPSLA |
| | | YLQSKGKNLGVINGRNYDDEDILKYVDVGATYYFNKNMST |
| | | YVDYKINLLDDNQFTRDAGINTDNIVALGLVYQF |
| SEQ ID NO: 4 | C-terminal domain of feline | DHPKQRGDAGSIQGAPKQTPDGGKPVKPLARVRRAPRGKP |
| | proliferation marker protein | VEDLAGHRDPVKSRSESSDSPSPKRKRGNEGGGPGTKRLR |
| | Ki67 | SVTPAQATAEEKPPLKKRRGAPREGRDPPEPLTAKRKLRV |
| | | AERMEVPEDLTSGKRESRTEGRGVGRTTASPQQAMSLRSR |
| | | RPNKAKLEEQRPEPVKATAEKVKTDRNDKKPWKTSQQTKP |
| | | QSPEGRAKSSTPAGRVLASRMCLRSTRPRKVPLPDVAEEK |
| | | QREKGVGVHVKNQEEEVTQRSDVMSLRSRKVKILPGGNAL |
| | | ESESQQRVTRSAKRCAGNVKKDEDNACVKKIRTRSRRDNE |
| | | DV |
| SEQ ID NO: 5 | C-terminal domain of feline | MGSSHHHHHHDHPKQRGDAGSIQGAPKQTPDGGKPVKPLA |
| | proliferation marker protein | RVRRAPRGKPVEDLAGHRDPVKSRSESSDSPSPKRKRGNE |
| | Ki67 with fusion tag | GGGPGTKRLRSVTPAQATAEEKPPLKKRRGAPREGRDPPE |
| | comprising a hexa-His | PLTAKRKLRVAERMEVPEDLTSGKRESRTEGRGVGRTTAS |
| | sequence | PQQAMSLRSRRPNKAKLEEQRPEPVKATAEKVKTDRNDKK |
| | | PWKTSQQTKPQSPEGRAKSSTPAGRVLASRMCLRSTRPRK |
| | | VPLPDVAEEKQREKGVGVHVKNQEEEVTQRSDVMSLRSRK |
| | | VKILPGGNALESESQQRVTRSAKRCAGNVKKDEDNACVKK |
| | | IRTRSRRDNEDV |
| SEQ ID NO: 6 | C-terminal domain of feline | MGSSHHHHHHGSWMSDSEVNQEAKPEVKPEVKPETHINLK |
| | proliferation marker protein | VSDGSSEIFFKIKKTTPLRRLMEAFAKRQGKEMDSLRFLY |
| | Ki67 with fusion tag, | DGIRIQADQTPEDLDMEDNDIIEAHREQIGGDHPKQRGDA |
| | comprising a hexa-His | GSIQGAPKQTPDGGKPVKPLARVRRAPRGKPVEDLAGHRD |
| | sequence and a SUMO tag | PVKSRSESSDSPSPKRKRGNEGGGPGTKRLRSVTPAQATA |
| | | EEKPPLKKRRGAPREGRDPPEPLTAKRKLRVAERMEVPED |
| | | LTSGKRESRTEGRGVGRTTASPQQAMSLRSRRPNKAKLEE |
| | | QRPEPVKATAEKVKTDRNDKKPWKTSQQTKPQSPEGRAKS |
| | | STPAGRVLASRMCLRSTRPRKVPLPDVAEEKQREKGVGVH |
| | | VKNQEEEVTQRSDVMSLRSRKVKILPGGNALESESQQRVT |
| | | RSAKRCAGNVKKDEDNACVKKIRTRSRRDNEDV |
| SEQ ID NO: 7 | N-terminal portion of | ELDAKISSPEKVTERGNPDLSLPGSCQPAPTCQKCILSHP |
| | extracellular region of | SCAWCKQLNFTASGVAEARRCGQRQELLAQGCPPGELEEP |
| | feline integrin beta subunit | RGWQEVLQDQPLGQGDRGEGATQLAPQQVRVTLRPGEPQQ |
| | | LRVRFLRAEGYPVDLYYLMDLSYSMKDDLERVRQLGQDLL |
| | | ARLQEVTHSVRIGFGSFVDKTVLPFVSTVPSKLRHPCPTR |
| | | LERCQSPFSFRHVLSLTSDAKAFEREVGRQSVSGNLDSPE |
| | | GGFDAILQAALCQEQIGWRNVSRLLVFTSDDTFHTAGDGK |
| | | LGGIFMPSDGHCHLDSDGLYSRSPEFDYPSVGQVAQALSA |
| | | ANIQPIFAVTSATLPVYQELSKLIPKSAVGELSEDSSNVV |
| | | QLIMDAYNSLSSTVTLEHSPLPPGVHISYESQCGGPEERE |
| | | REAGDRGQCNEVRINQTVIFLVTIQATHCLSEPHLLRLRA |
| | | LGFSEELTVELHTL |
| SEQ ID NO: 8 | N-terminal portion of | MGSSHHHHHHELDAKISSPEKVTERGNPDLSLPGSCQPAP |
| | extracellular region of | TCQKCILSHPSCAWCKQLNFTASGVAEARRCGQRQELLAQ |
| | feline integrin beta subunit | GCPPGELEEPRGWQEVLQDQPLGQGDRGEGATQLAPQQVR |
| | with fusion tag, comprising | VTLRPGEPQQLRVRFLRAEGYPVDLYYLMDLSYSMKDDLE |
| | a hexa-His sequence | RVRQLGQDLLARLQEVTHSVRIGFGSFVDKTVLPFVSTVP |
| | | SKLRHPCPTRLERCQSPFSFRHVLSLTSDAKAFEREVGRQ |
| | | SVSGNLDSPEGGFDAILQAALCQEQIGWRNVSRLLVFTSD |
| | | DTFHTAGDGKLGGIFMPSDGHCHLDSDGLYSRSPEFDYPS |
| | | VGQVAQALSAANIQPIFAVTSATLPVYQELSKLIPKSAVG |
| | | ELSEDSSNVVQLIMDAYNSLSSTVTLEHSPLPPGVHISYE |
| | | SQCGGPEEREREAGDRGQCNEVRINQTVIFLVTIQATHCL |
| | | SEPHLLRLRALGFSEELTVELHTL |
| SEQ ID NO: 9 | N-terminal portion of | MGSSHHHHHHGSGLVPRGSASMSDSEVNQEAKPEVKPEVK |
| | extracellular region of | PETHINLKVSDGSSEIFFKIKKTTPLRRLMEAFAKRQGKE |
| | feline integrin beta subunit | MDSLRFLYDGIRIQADQTPEDLDMEDNDIIEAHREQIGGE |
| | with fusion tag, comprising | LDAKISSPEKVTERGNPDLSLPGSCQPAPTCQKCILSHPS |
| | a hexa-His sequence, a | CAWCKQLNFTASGVAEARRCGQRQELLAQGCPPGELEEPR |
| | thrombin cleavage | GWQEVLQDQPLGQGDRGEGATQLAPQQVRVTLRPGEPQQL |
| | sequence and a SUMO | RVRFLRAEGYPVDLYYLMDLSYSMKDDLERVRQLGQDLLA |
| | sequence | RLQEVTHSVRIGFGSFVDKTVLPFVSTVPSKLRHPCPTRL |
| | | ERCQSPFSFRHVLSLTSDAKAFEREVGRQSVSGNLDSPEG |
| | | GFDAILQAALCQEQIGWRNVSRLLVFTSDDTFHTAGDGKL |
| | | GGIFMPSDGHCHLDSDGLYSRSPEFDYPSVGQVAQALSAA |
| | | NIQPIFAVTSATLPVYQELSKLIPKSAVGELSEDSSNVVQ |
| | | LIMDAYNSLSSTVTLEHSPLPPGVHISYESQCGGPEERER |
| | | EAGDRGQCNEVRINQTVIFLVTIQATHCLSEPHLLRLRAL |
| | | GFSEELTVELHTL |
| SEQ ID NO: | Feline keratin, type I | SFTTHSIFSSNYRSVGSVQGSNHRVRPVSSAASVYAGAGG |
| 10 | cytoskeletal 18 | SGSRISISRSSSFRSGWGSGGMAAVMTGALAGIGGIQGEK |
| | | ETMQDLNDRLASYLERVRSLEADNQRLEIKIREHLEKKGP |
| | | QVRDWGHYFKTIEELRAQIFANSVDNARIVLQIDNARLAA |
| | | DDFRVKYETELAMRQSVESDIHGLRKVIDDTNVTRLQLET |
| | | EIEALKEELLFMKKNHEEEVKGLQNQIANSGLTVELDAPK |
| | | SQDLSKIMADIRAQYEELAKKNREELDKYWSQQIEESTTV |
| | | ITSQTAEIAEAEKTLKELRRTVQSLEINLDSMRNLKASFE |
| | | NSLREVEARYTLQMEQLNGVLLHLESELSQTRTEGQRQAQ |
| | | EYEALLNVKVKLEAEIATYRRLLEDGEDFSLSDALDTSNS |
| | | FQTIQKTTTRRIVDGKVVSETNDTKVLR |
| SEQ ID NO: | Feline keratin, type I | MGSSHHHHHHSFTTHSIFSSNYRSVGSVQGSNHRVRPVSS |
| 11 | cytoskeletal 18 with a | AASVYAGAGGSGSRISISRSSSFRSGWGSGGMAAVMTGAL |
| | fusion tag | AGIGGIQGEKETMQDLNDRLASYLERVRSLEADNQRLEIK |
| | | IREHLEKKGPQVRDWGHYFKTIEELRAQIFANSVDNARIV |
| | | LQIDNARLAADDFRVKYETELAMRQSVESDIHGLRKVIDD |
| | | TNVTRLQLETEIEALKEELLFMKKNHEEEVKGLQNQIANS |
| | | GLTVELDAPKSQDLSKIMADIRAQYEELAKKNREELDKYW |
| | | SQQIEESTTVITSQTAEIAEAEKTLKELRRTVQSLEINLD |
| | | SMRNLKASFENSLREVEARYTLQMEQLNGVLLHLESELSQ |
| | | TRTEGQRQAQEYEALLNVKVKLEAEIATYRRLLEDGEDFS |
| | | LSDALDTSNSFQTIQKTTTRRIVDGKVVSETNDTKVLR |
| SEQ ID NO: | Feline keratin, type I | MGSSHHHHHHGSGLVPRGSASMSDSEVNQEAKPEVKPEVK |
| 12 | cytoskeletal 18 with fusion | PETHINLKVSDGSSEIFFKIKKTTPLRRLMEAFAKRQGKE |
| | tag comprising a hexa-His | MDSLRFLYDGIRIQADQTPEDLDMEDNDIIEAHREQIGGS |
| | sequence | FTTHSIFSSNYRSVGSVQGSNHRVRPVSSAASVYAGAGGS |
| | | GSRISISRSSSFRSGWGSGGMAAVMTGALAGIGGIQGEKE |
| | | TMQDLNDRLASYLERVRSLEADNQRLEIKIREHLEKKGPQ |
| | | VRDWGHYFKTIEELRAQIFANSVDNARIVLQIDNARLAAD |
| | | DFRVKYETELAMRQSVESDIHGLRKVIDDTNVTRLQLETE |
| | | IEALKEELLFMKKNHEEEVKGLQNQIANSGLTVELDAPKS |
| | | QDLSKIMADIRAQYEELAKKNREELDKYWSQQIEESTTVI |
| | | TSQTAEIAEAEKTLKELRRTVQSLEINLDSMRNLKASFEN |
| | | SLREVEARYTLQMEQLNGVLLHLESELSQTRTEGQRQAQE |
| | | YEALLNVKVKLEAEIATYRRLLEDGEDFSLSDALDTSNSF |
| | | QTIQKTTTRRIVDGKVVSETNDTKVLR |
| SEQ ID NO: | Example of a hexa-His (or | MGSSHHHHHH |
| 13 | 6xHIS) tag, including N- | |
| | terminal start sequence | |
| SEQ ID NO: | Example of a thrombin | LVPRGS |
| 14 | cleavage sequence | |
| SEQ ID NO: | Example of a SUMO | DSEVNQEAKPEVKPEVKPETHINLKVSDGSSEIFFKIKKT |
| 15 | sequence | TPLRRLMEAFAKRQGKEMDSLRFLYDGIRIQADQTPEDLD |
| | | MEDNDIIEAHREQIGG |
| SEQ ID NO: | Example of a SUMO | LVPRGSASMSDSEVNQEAKPEVKPEVKPETHINLKVSDGS |
| 16 | sequence with a thrombin | SEIFFKIKKTTPLRRLMEAFAKRQGKEMDSLRFLYDGIRI |
| | cleavage sequence | QADQTPEDLDMEDNDIIEAHREQIGG |
| SEQ ID NO: | Example of a hexa-His | MGSSHHHHHHGSGLVPRGSASMSDSEVNQEAKPEVKPEVK |
| 17 | sequence plus SUMO | PETHINLKVSDGSSEIFFKIKKTTPLRRLMEAFAKRQGKE |
| | sequence and N-terminal | MDSLRFLYDGIRIQADQTPEDLDMEDNDIIEAHREQIGG |
| | start sequence | |
| SEQ ID NO: | Example of feline TK1 C- | SVCRLCYFKKASGLPAGPDGKENKENCPLLGKPGEASGAR |
| 18 | terminal sequence | KLFAPHQILQCSSAN |
| SEQ ID NO: | Example of C-terminal | MGSSHHHHHHSVCRLCYFKKASGLPAGPDGKENKENCPLL |
| 19 | domain of feline | GKPGEASGARKLFAPHQILQCSSAN |
| | proliferation marker protein | |
| | TK1 with fusion tag | |
| | comprising a hexa-His | |
| | sequence | |
| SEQ ID NO: | Example of feline TK1 C- | MGSSHHHHHHGSGLVPRGSASMSDSEVNQEAKPEVKPEVK |
| 20 | terminal sequence with | PETHINLKVSDGSSEIFFKIKKTTPLRRLMEAFAKRQGKE |
| | fusion tag comprising hexa- | MDSLRFLYDGIRIQADQTPEDLDMEDNDIIEAHREQIGGS |
| | His sequence, SUMO | VCRLCYFKKASGLPAGPDGKENKENCPLLGKPGEASGARK |
| | sequence and N-terminal | LFAPHQILQCSSAN |
| | start sequence | |
| SEQ ID NO: | Example of feline TK1 C- | MGSSHHHHHHGSWMSDSEVNQEAKPEVKPEVKPETHINLK |
| 21 | terminal sequence with | VSDGSSEIFFKIKKTTPLRRLMEAFAKRQGKEMDSLRFLY |
| | fusion tag comprising hexa- | DGIRIQADQTPEDLDMEDNDIIEAHREQIGGGSVCRLCYF |
| | His sequence, SUMO | KKASGLPAGPDGKENKENCPLLGKPGEASGARKLFAPHQI |
| | sequence and N-terminal | LQCSSAN |
| | start sequence | |
| SEQ ID NO: | Example of feline TK1 C- | MGSSHHHHHHGSWMSDSEVNQEAKPEVKPEVKPETHINLK |
| 22 | terminal sequence with | VSDGSSEIFFKIKKTTPLRRLMEAFAKRQGKEMDSLRFLY |
| | fusion tag, comprising a | DGIRIQADQTPEDLDMEDNDIIEAHREQIGGSVCRLCYFK |
| | hexa-His sequence, SUMO | KASGLPAGPDGKENKENCPLLGKPGEASGARKLFAPHQIL |
| | sequence and N-termina | QCSSAN |
| | start sequence | |

1. A method for detecting endogenous IgA to a proliferation marker in serum obtained from a feline patient, wherein the proliferation marker is selected from Ki67 and TK1, the method comprising
a) contacting the serum with an antigen for a proliferation marker, selected from
a feline Ki67 antigen; and
a feline TK1 antigen; and
b) detecting endogenous IgA to the proliferation marker using a labeled antibody which binds feline IgA.

2. The method of claim 1 further comprising detecting detecting endogenous IgA to OmpC in serum obtained from a feline patient, wherein the OmpC corresponds to an OmpC from a feline microbiome, comprising
a) contacting the serum with an OmpC antigen; and
b) detecting endogenous IgA to OmpC using a labeled antibody which binds feline IgA.

3. The method of claim 1 further comprising detecting endogenous IgA to feline integrin or feline keratin in serum obtained from a feline patient, the method comprising
a) contacting the serum with an antigenic portion of a marker for wound repair or inflammation, selected from a feline integrin antigen and an antigen comprising feline keratin, type I cytoskeletal 18; and
b) detecting endogenous IgA to feline integrin or feline keratin using a labeled antibody which binds feline IgA.

4. (canceled)

5. The method of claim 1, further comprising determining the presence or level of additional endogenous antibodies in serum from the feline patient, wherein the additional endogenous antibodies are selected from one or more of endogenous antibodies to polymorphonuclear leukocytes (PMN), to calprotectin, to lactoferritin, to C-reactive protein, and to food sensitivity antigens.

6. The method of claim 1, comprising the steps of
a) affixing feline Ki67 antigen and/or feline TK1 antigen to a substrate,
b) blocking any uncoated surfaces of the substrates with protein,
c) exposing the antigens to the serum sample to allow formation of antigen-antibody complexes between the antigen and endogenous IgA,
d) exposing the antigen-IgA complexes thus formed to the labeled antibody,
e) detecting binding of the labeled antibody to the antigen-IgA complexes.

7. The method of claim 6 wherein the substrate is washed with buffer after each of steps a-d.

8. The method of claim 6 or 7 wherein the labeled antibody is an anti-feline IgA antibody linked to an enzyme.

9. The method of claim 8 comprising providing a substrate for the enzyme, and measuring the increase in optical density caused by the reaction of the enzyme with the substrate for the enzyme, wherein the increase in optical density correlates with the presence and amount of endogenous IgA bound to antigen.

10. The method of claim 9 wherein the enzyme is horseradish peroxidase (HRP) and the substrate is 3,3′,5,5′-Tetramethylbenzidine (TMB).

11. The method of claim 1 wherein the feline Ki67 antigen is a fusion protein comprising SEQ ID NO: 4 and comprising any one or more of SEQ ID NO. 13-17.

12. A The method of claim 1 wherein the feline TK1 antigen is a fusion protein comprising SEQ ID NO: 18 and further comprising any one or more of SEQ ID NO. 13-17.

13. The method of claim 2 wherein the OmpC antigen is a fusion protein comprising SEQ ID NO: 1 and further comprising any one or more of SEQ ID NO. 13-17.

14. The method of claim 3 wherein the feline integrin antigen which is a fusion protein comprising SEQ ID NO: 7 and comprising any one or more of SEQ ID NO. 13-17.

15. The method of claim 3 wherein the feline keratin antigen is a fusion protein comprising SEQ ID NO: 10 and comprising any one or more of SEQ ID NO. 13-17.

16. (canceled)

17. A method of treating a feline having serum levels of greater than 75 ELISA units per milliliter of endogenous IgA antibodies to one or more, or all, of OmpC (ACA), Ki67 (AKiA), TK1, integrin (AINTA) and keratin (AKERA), wherein the feline has gastrointestinal lymphoma, comprising administering to the feline an effective amount of a chemotherapeutic agent.

18. A method of treating a feline having serum levels of 25-75 ELISA units per milliliter of endogenous IgA antibodies to one or more or all of OmpC (ACA), Ki67 (AKiA), integrin (AINTA) and keratin (AKERA), wherein the feline is suffering from inflammatory bowel disease, comprising administering to the feline an effective amount of an anti-inflammatory agent.

19. (canceled)

20. (canceled)

21. A kit for use in a method according to claim 1, comprising
a) an antigen comprising a sequence selected from SEQ ID NOS: 1-12 and 18-22; and
b) a labeled antibody which binds feline IgA.


SEQUENCE LISTING (22 sequences)

SEQ ID NO: 1 - protein, 354 aa, Escherichia coli
  MISC_FEATURE: OmpC isolated from E. coli collected from cat biopsy
AEVYNKDGNKLDLYGKVDGLHYFSDDKSVDGDQTYMRLGFKGETQVTDQLTGYGQWEYQIQGNSAENENNSWTRVAFAGLKFQDVGSFDYGRNYGVVYDVTSWTDVLPEFGGDTYGSDNFMQQRGNGFATYRNTDFFGLVDGLNFAVQYQGKNGSVDGEGMTNNGRGALRQNGDGVGGSITYDYEGFGIGAAVSSSKRTDAQNGTYVVDNVTHNYIGTGDRAETYTGGLKYDANNIYLAAQYTQTYNATRVGSLGWANKAQNFEAVAQYQFDFGLRPSLAYLQSKGKNLGVINGRNYDDEDILKYVDVGATYYFNKNMSTYVDYKINLLDDNQFTRDAGINTDNIVALGLVYQF

SEQ ID NO: 2 - protein, 364 aa, Artificial Sequence
  OmpC with a fusion tag comprising a hexa-His sequence
MGSSHHHHHHAEVYNKDGNKLDLYGKVDGLHYFSDDKSVDGDQTYMRLGFKGETQVTDQLTGYGQWEYQIQGNSAENENNSWTRVAFAGLKFQDVGSFDYGRNYGVVYDVTSWTDVLPEFGGDTYGSDNFMQQRGNGFATYRNTDFFGLVDGLNFAVQYQGKNGSVDGEGMTNNGRGALRQNGDGVGGSITYDYEGFGIGAAVSSSKRTDAQNGTYVVDNVTHNYIGTGDRAETYTGGLKYDANNIYLAAQYTQTYNATRVGSLGWANKAQNFEAVAQYQFDFGLRPSLAYLQSKGKNLGVINGRNYDDEDILKYVDVGATYYFNKNMSTYVDYKINLLDDNQFTRDAGINTDNIVALGLVYQF

SEQ ID NO: 3 - protein, 474 aa, Artificial Sequence
  OmpC with a fusion tag comprising a hexa-His sequence, a thrombin cleavage sequence, and a SUMO sequence.
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGRAEVYNKDGNKLDLYGKVDGLHYFSDDKSVDGDQTYMRLGFKGETQVTDQLTGYGQWEYQIQGNSAENENNSWTRVAFAGLKFQDVGSFDYGRNYGVVYDVTSWTDVLPEFGGDTYGSDNFMQQRGNGFATYRNTDFFGLVDGLNFAVQYQGKNGSVDGEGMTNNGRGALRQNGDGVGGSITYDYEGFGIGAAVSSSKRTDAQNGTYVVDNVTHNYIGTGDRAETYTGGLKYDANNIYLAAQYTQTYNATRVGSLGWANKAQNFEAVAQYQFDFGLRPSLAYLQSKGKNLGVINGRNYDDEDILKYVDVGATYYFNKNMSTYVDYKINLLDDNQFTRDAGINTDNIVALGLVYQF

SEQ ID NO: 4 - protein, 322 aa, Felis catus
  MISC_FEATURE (1)..(322): C-terminal domain of feline proliferation marker protein Ki67
DHPKQRGDAGSIQGAPKQTPDGGKPVKPLARVRRAPRGKPVEDLAGHRDPVKSRSESSDSPSPKRKRGNEGGGPGTKRLRSVTPAQATAEEKPPLKKRRGAPREGRDPPEPLTAKRKLRVAERMEVPEDLTSGKRESRTEGRGVGRTTASPQQAMSLRSRRPNKAKLEEQRPEPVKATAEKVKTDRNDKKPWKTSQQTKPQSPEGRAKSSTPAGRVLASRMCLRSTRPRKVPLPDVAEEKQREKGVGVHVKNQEEEVTQRSDVMSLRSRKVKILPGGNALESESQQRVTRSAKRCAGNVKKDEDNACVKKIRTRSRRDNEDV

SEQ ID NO: 5 - protein, 332 aa, Artificial Sequence
  C-terminal domain of feline proliferation marker protein Ki67 with fusion tag comprising a hexa-His sequence
MGSSHHHHHHDHPKQRGDAGSIQGAPKQTPDGGKPVKPLARVRRAPRGKPVEDLAGHRDPVKSRSESSDSPSPKRKRGNEGGGPGTKRLRSVTPAQATAEEKPPLKKRRGAPREGRDPPEPLTAKRKLRVAERMEVPEDLTSGKRESRTEGRGVGRTTASPQQAMSLRSRRPNKAKLEEQRPEPVKATAEKVKTDRNDKKPWKTSQQTKPQSPEGRAKSSTPAGRVLASRMCLRSTRPRKVPLPDVAEEKQREKGVGVHVKNQEEEVTQRSDVMSLRSRKVKILPGGNALESESQQRVTRSAKRCAGNVKKDEDNACVKKIRTRSRRDNEDV

SEQ ID NO: 6 - protein, 433 aa, Artificial Sequence
  C-terminal domain of feline proliferation marker protein Ki67 with fusion tag, comprising a hexa-His sequence and a SUMO tag
MGSSHHHHHHGSWMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGDHPKQRGDAGSIQGAPKQTPDGGKPVKPLARVRRAPRGKPVEDLAGHRDPVKSRSESSDSPSPKRKRGNEGGGPGTKRLRSVTPAQATAEEKPPLKKRRGAPREGRDPPEPLTAKRKLRVAERMEVPEDLTSGKRESRTEGRGVGRTTASPQQAMSLRSRRPNKAKLEEQRPEPVKATAEKVKTDRNDKKPWKTSQQTKPQSPEGRAKSSTPAGRVLASRMCLRSTRPRKVPLPDVAEEKQREKGVGVHVKNQEEEVTQRSDVMSLRSRKVKILPGGNALESESQQRVTRSAKRCAGNVKKDEDNACVKKIRTRSRRDNEDV

SEQ ID NO: 7 - protein, 454 aa, Felis catus
  MISC_FEATURE (1)..(454): N-terminal portion of extracellular region of feline integrin beta subunit
ELDAKISSPEKVTERGNPDLSLPGSCQPAPTCQKCILSHPSCAWCKQLNFTASGVAEARRCGQRQELLAQGCPPGELEEPRGWQEVLQDQPLGQGDRGEGATQLAPQQVRVTLRPGEPQQLRVRFLRAEGYPVDLYYLMDLSYSMKDDLERVRQLGQDLLARLQEVTHSVRIGFGSFVDKTVLPFVSTVPSKLRHPCPTRLERCQSPFSFRHVLSLTSDAKAFEREVGRQSVSGNLDSPEGGFDAILQAALCQEQIGWRNVSRLLVFTSDDTFHTAGDGKLGGIFMPSDGHCHLDSDGLYSRSPEFDYPSVGQVAQALSAANIQPIFAVTSATLPVYQELSKLIPKSAVGELSEDSSNVVQLIMDAYNSLSSTVTLEHSPLPPGVHISYESQCGGPEEREREAGDRGQCNEVRINQTVIFLVTIQATHCLSEPHLLRLRALGFSEELTVELHTL

SEQ ID NO: 8 - protein, 464 aa, Artificial Sequence
  N-terminal portion of extracellular region of feline integrin beta subunit with fusion tag, comprising a hexa-His sequence
MGSSHHHHHHELDAKISSPEKVTERGNPDLSLPGSCQPAPTCQKCILSHPSCAWCKQLNFTASGVAEARRCGQRQELLAQGCPPGELEEPRGWQEVLQDQPLGQGDRGEGATQLAPQQVRVTLRPGEPQQLRVRFLRAEGYPVDLYYLMDLSYSMKDDLERVRQLGQDLLARLQEVTHSVRIGFGSFVDKTVLPFVSTVPSKLRHPCPTRLERCQSPFSFRHVLSLTSDAKAFEREVGRQSVSGNLDSPEGGFDAILQAALCQEQIGWRNVSRLLVFTSDDTFHTAGDGKLGGIFMPSDGHCHLDSDGLYSRSPEFDYPSVGQVAQALSAANIQPIFAVTSATLPVYQELSKLIPKSAVGELSEDSSNVVQLIMDAYNSLSSTVTLEHSPLPPGVHISYESQCGGPEEREREAGDRGQCNEVRINQTVIFLVTIQATHCLSEPHLLRLRALGFSEELTVELHTL

SEQ ID NO: 9 - protein, 573 aa, Artificial Sequence
  N-terminal portion of extracellular region of feline integrin beta subunit with fusion tag, comprising a hexa-His sequence, a thrombin cleavage sequence and a SUMO sequence
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGELDAKISSPEKVTERGNPDLSLPGSCQPAPTCQKCILSHPSCAWCKQLNFTASGVAEARRCGQRQELLAQGCPPGELEEPRGWQEVLQDQPLGQGDRGEGATQLAPQQVRVTLRPGEPQQLRVRFLRAEGYPVDLYYLMDLSYSMKDDLERVRQLGQDLLARLQEVTHSVRIGFGSFVDKTVLPFVSTVPSKLRHPCPTRLERCQSPFSFRHVLSLTSDAKAFEREVGRQSVSGNLDSPEGGFDAILQAALCQEQIGWRNVSRLLVFTSDDTFHTAGDGKLGGIFMPSDGHCHLDSDGLYSRSPEFDYPSVGQVAQALSAANIQPIFAVTSATLPVYQELSKLIPKSAVGELSEDSSNVVQLIMDAYNSLSSTVTLEHSPLPPGVHISYESQCGGPEEREREAGDRGQCNEVRINQTVIFLVTIQATHCLSEPHLLRLRALGFSEELTVELHTL

SEQ ID NO: 10 - protein, 428 aa, Felis catus
  MISC_FEATURE (1)..(428): Feline keratin, type I cytoskeletal 18
SFTTHSIFSSNYRSVGSVQGSNHRVRPVSSAASVYAGAGGSGSRISISRSSSFRSGWGSGGMAAVMTGALAGIGGIQGEKETMQDLNDRLASYLERVRSLEADNQRLEIKIREHLEKKGPQVRDWGHYFKTIEELRAQIFANSVDNARIVLQIDNARLAADDFRVKYETELAMRQSVESDIHGLRKVIDDTNVTRLQLETEIEALKEELLFMKKNHEEEVKGLQNQIANSGLTVELDAPKSQDLSKIMADIRAQYEELAKKNREELDKYWSQQIEESTTVITSQTAEIAEAEKTLKELRRTVQSLEINLDSMRNLKASFENSLREVEARYTLQMEQLNGVLLHLESELSQTRTEGQRQAQEYEALLNVKVKLEAEIATYRRLLEDGEDFSLSDALDTSNSFQTIQKTTTRRIVDGKVVSETNDTKVLR

SEQ ID NO: 11 - protein, 438 aa, Artificial Sequence
  Feline keratin, type I cytoskeletal 18 with a fusion tag
MGSSHHHHHHSFTTHSIFSSNYRSVGSVQGSNHRVRPVSSAASVYAGAGGSGSRISISRSSSFRSGWGSGGMAAVMTGALAGIGGIQGEKETMQDLNDRLASYLERVRSLEADNQRLEIKIREHLEKKGPQVRDWGHYFKTIEELRAQIFANSVDNARIVLQIDNARLAADDFRVKYETELAMRQSVESDIHGLRKVIDDTNVTRLQLETEIEALKEELLFMKKNHEEEVKGLQNQIANSGLTVELDAPKSQDLSKIMADIRAQYEELAKKNREELDKYWSQQIEESTTVITSQTAEIAEAEKTLKELRRTVQSLEINLDSMRNLKASFENSLREVEARYTLQMEQLNGVLLHLESELSQTRTEGQRQAQEYEALLNVKVKLEAEIATYRRLLEDGEDFSLSDALDTSNSFQTIQKTTTRRIVDGKVVSETNDTKVLR

SEQ ID NO: 12 - protein, 547 aa, Artificial Sequence
  Feline keratin, type I cytoskeletal 18 with fusion tag comprising a hexa-His sequence
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSFTTHSIFSSNYRSVGSVQGSNHRVRPVSSAASVYAGAGGSGSRISISRSSSFRSGWGSGGMAAVMTGALAGIGGIQGEKETMQDLNDRLASYLERVRSLEADNQRLEIKIREHLEKKGPQVRDWGHYFKTIEELRAQIFANSVDNARIVLQIDNARLAADDFRVKYETELAMRQSVESDIHGLRKVIDDTNVTRLQLETEIEALKEELLFMKKNHEEEVKGLQNQIANSGLTVELDAPKSQDLSKIMADIRAQYEELAKKNREELDKYWSQQIEESTTVITSQTAEIAEAEKTLKELRRTVQSLEINLDSMRNLKASFENSLREVEARYTLQMEQLNGVLLHLESELSQTRTEGQRQAQEYEALLNVKVKLEAEIATYRRLLEDGEDFSLSDALDTSNSFQTIQKTTTRRIVDGKVVSETNDTKVLR

SEQ ID NO: 13 - protein, 10 aa, Artificial Sequence
  Example of a hexa-His (or 6xHIS) tag, including N-terminal start sequence
MGSSHHHHHH

SEQ ID NO: 14 - protein, 6 aa, Artificial Sequence
  Example of a thrombin cleavage sequence
LVPRGS

SEQ ID NO: 15 - protein, 96 aa, Artificial Sequence
  Example of a SUMO sequence
DSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 16 - protein, 106 aa, Artificial Sequence
  Example of a SUMO sequence with a thrombin cleavage sequence
LVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 17 - protein, 119 aa, Artificial Sequence
  Example of a hexa-His sequence plus SUMO sequence and N-terminal start sequence
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGG

SEQ ID NO: 18 - protein, 55 aa, Felis catus
  MISC_FEATURE (1)..(55): Example of feline TK1 C-terminal sequence
SVCRLCYFKKASGLPAGPDGKENKENCPLLGKPGEASGARKLFAPHQILQCSSAN

SEQ ID NO: 19 - protein, 65 aa, Artificial Sequence
  Example of C-terminal domain of feline proliferation marker protein TK1 with fusion tag comprising a hexa-His sequence
MGSSHHHHHHSVCRLCYFKKASGLPAGPDGKENKENCPLLGKPGEASGARKLFAPHQILQCSSAN

SEQ ID NO: 20 - protein, 174 aa, Artificial Sequence
  Example of feline TK1 C-terminal sequence with fusion tag comprising hexa-His sequence, SUMO sequence and N-terminal start sequence
MGSSHHHHHHGSGLVPRGSASMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSVCRLCYFKKASGLPAGPDGKENKENCPLLGKPGEASGARKLFAPHQILQCSSAN

SEQ ID NO: 21 - protein, 167 aa, Artificial Sequence
  Example of feline TK1 C-terminal sequence with fusion tag comprising hexa-His sequence, SUMO sequence and N-terminal start sequence
MGSSHHHHHHGSWMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGGSVCRLCYFKKASGLPAGPDGKENKENCPLLGKPGEASGARKLFAPHQILQCSSAN

SEQ ID NO: 22 - protein, 166 aa, Artificial Sequence
  Example of feline TK1 C-terminal sequence with fusion tag, comprising a hexa-His sequence, SUMO sequence and N-terminal start sequence
MGSSHHHHHHGSWMSDSEVNQEAKPEVKPEVKPETHINLKVSDGSSEIFFKIKKTTPLRRLMEAFAKRQGKEMDSLRFLYDGIRIQADQTPEDLDMEDNDIIEAHREQIGGSVCRLCYFKKASGLPAGPDGKENKENCPLLGKPGEASGARKLFAPHQILQCSSAN